(19) United States
(12) Patent Application Publication
(10) Pub. No.: US 2005/0256039 A1
(43) Pub. Date: Nov. 17, 2005

(54) Novel fibroblast growth factors and methods of use thereof

(76) Inventors: Jeffery Peterson, Guilford, CT (US); Paul Sciore, Calgary (CA); Peter Mezes, Old Lyme, CT (US); Henri Lichenstein, Guilford, CT (US); Michael Jeffers, Branford, CT (US); William LaRochelle, Madison, CT (US); Marie Ruiz-Martinez, Bethany, CT (US); Galina Chernaya, Madison, CT (US)

Correspondence Address: CURAGEN CORPORATION, 322 EAST MAIN STREET, BRANFORD, CT, 06405, US

(21) Appl. No.: 10/842,206

(22) Filed: May 10, 2004

Related U.S. Application Data
(60) Provisional application No. 60/469,353, filed on May 9, 2003.

(51) Int. Cl. A61K 38/17; C07H 21/04; C12P 21/06; C12N 15/09; C07K 14/47

(52) U.S. Cl. 514/12; 435/69.1; 435/320.1; 435/325; 530/350; 435/252.3; 536/23.5

(57) ABSTRACT

The present invention relates to compositions and methods of treatment of various conditions, including but are not limited to, stroke, wound healing, and joint diseases (e.g., osteoarthritis and rheumatoid arthritis). More particularly, the present invention relates to compositions comprising a member of the fibroblast growth factor family, FGF-CX (also known as CG53135-05 or FGF-20), its related polypeptides, nucleic acids encoding such polypeptides, and their uses for treating a condition, such as but is not limited to, stroke, would healing, and joint diseases (e.g., osteoarthritis and rheumatoid arthritis).

This application claims the benefit of U.S. Provisional Application Ser. No. 60/469,353, filed May 9, 2003, which is incorporated herein by reference in its entirety.

### 1. FIELD OF INVENTION

The present invention relates to compositions and methods of treatment of various conditions, including but are not limited to, stroke, wound healing, and joint diseases (e.g., osteoarthritis and rheumatoid arthritis). More particularly, the present invention relates to compositions comprising a member of the fibroblast growth factor family, FGF-CX (also known as CG53135-05 or FGF-20), its related polypeptides, nucleic acids encoding such polypeptides, and their uses for treating a condition, such as but is not limited to, stroke, would healing, and joint diseases (e.g., osteoarthritis and rheumatoid arthritis).

### 2. BACKGROUND OF THE INVENTION

The FGF family of proteins, whose prototypic members include acidic FGF (FGF-1) and basic FGF (FGF-2), bind to four related receptor tyrosine kinases. These FGF receptors are expressed on most types of cells in tissue culture. Dimerization of FGF receptor monomers upon ligand binding has been reported to be a requisite for activation of the kinase domains, leading to receptor trans phosphorylation. FGF receptor-1 (FGFR-1), which shows the broadest expression pattern of the four FGF receptors, contains at least seven tyrosine phosphorylation sites. A number of signal transduction molecules are affected by binding with different affinities to these phosphorylation sites.

Expression of FGFs and their receptors in brains of perinatal and adult mice has been examined. Messenger RNA of all FGF genes, with the exception of FGF-4, is detected in these tissues. FGF-3, FGF-6, FGF-7 and FGF-8 genes demonstrate higher expression in the late embryonic stages than in postnatal stages, suggesting that these members are involved in the late stages of brain development. In contrast, expression of FGF-1 and FGF-5 increased after birth. In particular, FGF-6 expression in perinatal mice has been reported to be restricted to the central nervous system and skeletal muscles, with intense signals in the developing cerebrum in embryos but in cerebellum in 5-day-old neonates. FGF-receptor (FGFR)-4, a cognate receptor for FGF-6, demonstrates similar spatiotemporal expression, suggesting that FGF-6 and FGFR-4 play significant roles in the maturation of nervous system as a ligand-receptor system. According to Ozawa et al., these results strongly suggest that the various FGFs and their receptors are involved in the regulation of a variety of developmental processes of brain, such as proliferation and migration of neuronal progenitor cells, neuronal and glial differentiation, neurite extensions, and synapse formation. See, e.g., Ozawa et al., Brain Res. Mol. Brain Res. 1996 41(1-2):279-88.

Other members of the FGF polypeptide family include the FGF receptor tyrosine kinase (FGFRTK) family and the FGF receptor heparan sulfate proteoglycan (FGFRHS) family. These members interact to regulate active and specific FGFR signal transduction complexes. These regulatory activities are diversified throughout a broad range of organs and tissues, and in both normal and tumor tissues, in mammals. Regulated alternative messenger RNA (mRNA) splicing and combination of variant subdomains give rise to diversity of FGFRTK monomers. Divalent cations cooperate with the FGFRHS to conformationally restrict FGFRTK trans-phosphorylation, which causes depression of kinase activity and facilitates appropriate activation of the FGFR complex by FGF. For example, it is known that different point mutations in the FGFRTK commonly cause craniofacial and skeletal abnormalities of graded severity by graded increases in FGF-independent activity of total FGFR complexes. Other processes in which FGF family exerts important effects are liver growth and function, and prostate tumor progression.

Glia-activating factor (GAF), another FGF family member, is a heparin-binding growth factor that was purified from the culture supernatant of a human glioma cell line. See, Miyamoto et al., 1993, Mol. Cell Biol. 13(7): 42514259. GAF shows a spectrum of activity slightly different from those of other known growth factors, and is designated as FGF-9. The human FGF-9 cDNA encodes a polypeptide of 208 amino acids. Sequence similarity to other members of the FGF family was estimated to be around 30%. Two cysteine residues and other consensus sequences found in other family members were also well conserved in the FGF-9 sequence. FGF-9 was found to have no typical signal sequence in its N-terminus like those in acidic FGF and basic FGF. Acidic FGF and basic FGF are known not to be secreted from cells in a conventional manner. However, FGF-9 was found to be secreted efficiently from cDNA-transfected COS cells despite its lack of a typical signal sequence. It could be detected exclusively in the culture medium of cells. The secreted protein lacked no amino acid residues at the N-terminus with respect to those predicted by the cDNA sequence, except the initiation methionine. The rat FGF-9 cDNA was also cloned, and the structural analysis indicated that the FGF-9 gene is highly conserved.

FGFs have been shown to induce neuronal sprouting. See Proc. Natl. Acad. Sci. U.S.A. 1997 94 (15): 8179-84. Kawamata et al. (Journal of Cerebral Blood Flow and Metabolism, 16:542-547, 1996) proposed that basic FGF, an 18 kDa and 154 amino acid long polypeptide, supports the survival and outgrowth of a wide variety of brain neurons. US patent application U.S. 2002/0151496 A1 suggests that FGF-20 is a neurotrophic factor and stimulate survival of cells of neuronal origin.

2.1. Inflammation: Osteoarthritis and Rheumatoid Arthritis

Osteoarthritis (“OA”) is a degenerative joint disease and a frequent cause of joint pain that affects a large and growing population. OA is estimated to be the most common cause of disability in adults. The disease typically manifests itself in the 2nd to 3rd decades, with most people over forty years exhibiting some pathologic change in weight bearing joints, although the change may be asymptomatic. A systematic review of incidence and prevalence of OA of the knee in people older than 55 years in the United Kingdom reported an incidence of 25 percent per year, a prevalence of disability of 10 percent, and severe disability in about two to three percent. The National Health and Nutrition Examination Survey (Center for Health Statistics, Centers for Disease Control and Prevention) found the prevalence of this disease to be over 80 percent in people over age 55, compared to less than 0.1 percent in those aged 25 to 34 years old. OA results from a complex interplay of multiple factors, including joint integrity, genetics, local inflammation, mechanical forces, and cellular and biochemical processes. Characteristic features of the disease are degradation of articular cartilage, hypertrophy of bone at the margins, and changes in the synovial membrane, typically accompanied by pain and stiffness of the joint. For the majority of patients, OA is linked to one or more factors, such as aging, occupation, trauma, and repetitive and small insults over time. The pathophysiologic process of OA is almost always progressive.

CG53135-05 and their variants belong to the FGF family that regulates proliferation (see U.S. application Ser. No. 10/174,394, which is incorporated herein by its entirety). The identification of a polymorphism (CG53135-12) in the gene encoding an FGF20-like protein, CG53135-01, in humans, and a method for identifying individuals who are carriers of the genetic risk-altering factor for OA have been described in U.S. application Ser. No. 10/702,126 (“the '126 application”), which is incorporated herein by its entirety. The '126 application describes a DNA-based diagnostic test for identifying individuals with increased risk for OA and resultant musculoskeletal complications.

There are several well-established treatment modes for OA, ranging from non-pharmaceutical to pharmaceutical intervention. Non-pharmaceutical interventions include behaviour modification, weight loss, exercise, walking aids, avoidance of aggravating activities, as well as joint irrigation, and arthroscopic and surgical interventions. Current pharmaceutical interventions include nonsteroidal antiinflammatory drugs, intraarticular corticosteroids, and colchicine. In addition, FGF-18 has been shown to repair damaged cartilage in a rat meniscal tear model for OA (See Paper #0199, 50th Annual Meeting of the Orthopaedic Research Society, San Francisco Calif., 2004).

However, satisfactory treatment of OA is an unmet medical need, as existing therapeutics have not been successful in curtailing the incidence or the severity of the disease. Consequently, a therapeutic that can successfully treat osteoarthritis has the beneficial effects of decreasing morbidity, while potentially saving the healthcare system millions of dollars in costs associated with invasive surgical procedures, disability and ancillary support services.

Citation or discussion of a reference herein shall not be construed as an admission that such is prior art to the present invention.

### 3. SUMMARY OF THE INVENTION

The present invention provides methods of preventing or treating a disease (e.g., stroke, joint diseases, and trauma) comprising administering to a subject in need thereof a FGF-CX polypeptide, which has homology to Fibroblast Growth Factor (FGF) protein. The present invention also encompasses FGF-CX polynucleotide sequences and the FGF-CX polypeptides encoded by these nucleic acid sequences, and fragments, homologs, analogs, and derivatives thereof.

In accordance with the present invention, the diseases to be prevented or treated include, but are not limited to, joint diseases (non-limiting examples being arthritis, osteoarthritis, joint pathology, ligament and tendon injuries, and meniscal injuries), ischemic stroke, hemorrhagic stroke, trauma, spinal cord damage, heavy metal or toxin poisoning, and neurodegenerative diseases (non-limiting examples being Alzheimer's, Parkinson's Disease, Amyotrophic Lateral Sclerosis, Huntington's Disease).

In one aspect, the invention encompasses an isolated FGF-CX nucleic acid (SEQ ID NOs:1, 3, 5, 7, 9, 11, 13, 15, 17, 19, 21, 23, 25, 27, 29, 31, 33, 35, as shown in Table A), that encodes a FGF-CX polypeptide, or a fragment, homolog, analog or derivative thereof. The nucleic acid can include, but not limited to, nucleic acid sequence encoding a polypeptide at least 85% identical to a polypeptide comprising the amino acid sequence of Table A (SEQ ID NOs: 2, 4, 6, 8, 10, 12, 14, 16, 18, 20, 22, 24, 26, 28, 30, 32, 34, 36). The invention also encompasses the polypeptides resulting from the proteolytic cleavage of CG53135-05 (SEQ ID NO: 2) that includes SEQ ID Nos: 37, 38, 39, 40. The nucleic acid can be, but is not limited to, a genomic DNA fragment, and a cDNA molecule.

The present invention also encompasses a vector containing one or more of the nucleic acids described herein, and a cell containing the vectors or nucleic acids described herein.

The present invention further encompasses host cells transformed with a recombinant expression vector comprising any of the nucleic acid molecules described above.

In one embodiment, the invention provides a pharmaceutical composition that comprises a FGF-CX nucleic acid and a pharmaceutically acceptable carrier. In another embodiment, the invention provides a substantially purified FGF-CX polypeptide, e.g., any of the FGF-CX polypeptides encoded by a FGF-CX nucleic acid, and fragments, homologs, analogs, and derivatives thereof. The invention also provides a pharmaceutical composition that comprises a FGF-CX polypeptide and a pharmaceutically acceptable carrier.

In another embodiment, the invention provides an antibody that binds specifically to a FGF-CX polypeptide. The antibody can be, but is not limited to, a monoclonal or polyclonal antibody, and fragments, homologs, analogs, and derivatives thereof. The invention also provides a pharmaceutical composition including FGF-CX antibody and a pharmaceutically acceptable carrier. The present invention also emcompasses isolated antibodies that bind to an epitope on a polypeptide encoded by any of the nucleic acid molecules described above.

The present invention further provides kits comprising antibodies that bind to a polypeptide encoded by any of the nucleic acid molecules described above and a negative control antibody.

The invention encompasses a method for producing a FGF-CX polypeptide. The method includes providing a cell containing a FGF-CX nucleic acid, e.g., a vector that includes a FGF-CX nucleic acid, and culturing the cell under conditions sufficient to express the FGF-CX polypeptide encoded by the nucleic acid. The expressed FGF-CX polypeptide is then recovered from the cell. Preferably, the cell produces little or no endogenous FGF-CX polypeptide. The cell can be, e.g., a prokaryotic cell or eukaryotic cell.

The present invention provides a method of inducing an immune response in a subject against a polypeptide encoded by any of the nucleic acid molecules disclosed above by administering to the mammal an amount of the polypeptide sufficient to induce the immune response.

The present invention also provides methods of identifying a compound that binds to FGF-CX polypeptide by contacting the FGF-CX polypeptide with a compound and determining whether the compound binds to the FGF-CX polypeptide.

The invention provides a prophylactic treatment with FGF-CX polypeptide wherein an injury that predisposes the subject to osteoarthritis has occurred but the cartilage is intact.

The invention also provides a therapeutic treatment with FGF-CX polypeptide wherein intrinsic or extrinsic factors (e.g. genetic predisposition or meniscal injury, respectively) has led to osteoarthritic changes and cartilage damage.

### 4. BRIEF DESCRIPTION OF THE FIGURES

FIG. 1 shows Liquid Chromatography and Mass Spectrometry analysis of CG53135-05. CG53135-05 was injected onto the phenyl-hexyl column in an aqueous mobile phase containing 95% water, 5% acetonitrile, and 0.1% trifluoroacetic acid. The protein was then eluted by using a non-linear gradient with an organic mobile phase containing 95% acetonitrile, 5% water, and 0.085% trifluoroacetic acid. Each of the 4 peaks was characterized using LC/ESI/MS, MALDI-TOF MS, and N-terminal amino acid sequencing.

FIGS. 2A and 2B depict Peptide Map of CG53135-05. The upper tracing in each panel represents that of CG53135-05 and the lower tracing in each panel represents an identical sample treated similarly but without CG53135-05. FIG. 2A: Detection at 214 nm to monitor CG53135 peptides. FIG. 2B: Detection at 295 nm to monitor tryptophan-containing peptides.

FIG. 3 shows Receptor Binding Specificity of CG53135. NIH 3T3 cells were serum-starved, incubated with the indicated factor (green squares=platelet derived growth factor; blue triangle=FGF-1; red circle=CG53135) either alone or together with the indicated soluble FGFR, and DNA synthesis in response to CG53135 was measured in a BrdU incorporation assay. Data points represent the average obtained from triplicate wells, and are represented as the percent BrdU incorporation relative to cells receiving factor alone.

FIG. 4 shows the results of Forelimb Placing Test. The mean and standard error of the score for groups receiving vehicle (diamonds), 1.0 μg/injection CG53135-05 (square), and 2.5 μg/injection CG53135-05 (triangles) are represented over time. Asterisks indicate significant difference from vehicle control as assessed by one-way ANOVA.

FIG. 5 shows the results of Hindlimb Placing Test. The mean and standard error of the score for groups receiving vehicle (diamonds), 1.0 μg/injection CG53135-05 (square), and 2.5 μg/injection CG53135-05 (triangles) are represented over time. Asterisks indicate significant difference from vehicle control as assessed by one-way ANOVA.

FIG. 6 shows the results of Body Swing Test. The mean and standard error of the score for groups receiving vehicle (diamonds), 1.0 μg/injection CG53135-05 (square), and 2.5 μg/injection CG53135-05 (triangles) are represented over time. A score range of ˜50% swings to the right indicates no impairment, whereas 0% swings to the right swing indicates maximal impairment. Asterisks indicate significant difference from vehicle control as assessed by one-way ANOVA.

FIG. 7 shows the results of Cylinder Test. The mean and standard error of the score for groups receiving vehicle (diamonds), 1.0 μg/injection CG53135-05 (square), and 2.5 μg/injection CG53135-05 (triangles) are represented over time.

FIG. 8 shows the results of Body Weight. The mean and standard errors of the weights for groups receiving vehicle (diamonds), 1.0 μg/injection CG53135-05 (square), and 2.5 μg/injection CG53135-05 (triangles) is represented over time.

FIG. 9 shows the effect of CG53135-05 on Pro-MMP production in SW1353 cells in the presence of IL-1 beta.

FIG. 10 shows the effect of CG53135-05 on Pro-MMP production in SW1353 cells in the presence of TNF-alpha.

FIG. 11 shows the effect of CG53135-05 on TIMP production in SW1353 cells.

FIG. 12 shows the effect of intra-articular injection of CG53135-05 in the Meniscal Tear Model of Rat Osteoarthritis (Prophylactic Dosing): Mean Tibial Cartilage Degeneration.

FIG. 13 shows results of intra-articular injection of CG53135-05 in the Meniscal Tear Model of Rat Osteoarthritis: (Prophylactic Dosing): Total Cartilage Degeneration Width.

FIG. 14 shows results of intra-articular injection of CG53135-05 in the Meniscal Tear Model of Rat Osteoarthritis: (Prophylactic Dosing): Significant Tibial Cartilage Degeneration Width.

FIG. 15 shows results of intra-articular injection CG53135-05 in the Meniscal Tear Model of Rat Osteoarthritis (Therapeutic Dosing): Mean Tibial Degeneration.

FIG. 16 shows results of intra-articular injection of CG53135-05 in the Meniscal Tear Model of Rat Osteoarthritis (Therapeutic Dosing): Total Cartilage Degeneration Width.

FIG. 17 shows results of intra-articular injection of CG53135-05 in Meniscal Tear Model of Rat Osteoarthritis (Therapeutic Dosing): Significant Tibial Cartilage Degeneration Width.

### 5. DETAILED DESCRIPTION OF THE INVENTION

The present invention provides methods of preventing or treating a joint disease (e.g., osteoarthritis, other related joint pathologies, such as but are not limited to, ligament and tendon injuries within the ligament and tendon itself, or within their respective insertion sites, meniscal tears, other joint disorders where matrix deposition occurs, joint disorders where remodeling and repair are required, and cartilage and joint pathology occurred as a result of an inflammatory disease (e.g., rheumatoid arthritis)) in a subject comprising administering to the subject a composition comprising an FGF-CX polypeptide.

The present invention also encompasses methods of using FGF-CX to improve functional recovery following middle cerebral artery (MCA) occlusion in rats. As stroke may result in disturbances of motor strength and coordination, sensory discrimination, visual function, speech, memory or other intellectual abilities, the present invention evaluates the efficacy and safety of FGF-CX in a model that assesses these parameters. In accordance with the present invention, administering FGF-CX will be beneficial in the treatment of pathological conditions including, but are not limited to, ischemic stroke, hemorrhagic stroke, trauma, spinal cord damage, heavy metal or toxin poisoning, and neurodegenerative diseases (such as Alzheimer's, Parkinson's Disease, Amyotrophic Lateral Sclerosis, Huntington's Disease).

FGF-CX polypeptides, nucleic acids encoding the polypeptides, and methods of making such polypeptides are described in U.S. application Ser. Nos. 09/494,585 and 10/174,394, both of which are incorporated herein by reference in their entireties. FGF-CX is used interchangeably with the term “CG53135,” “CG53135-05,” and “FGF-20.”

Included within the invention are FGF-CX nucleic acids, isolated nucleic acids that encode FGF-CX polypeptide or a portion thereof, FGF-CX polypeptides, vectors containing these nucleic acids, host cells transformed with the FGF-CX nucleic acids, anti-FGF-CX antibodies, and pharmaceutical compositions. Also disclosed are methods of making FGF-CX polypeptides, as well as methods of screening, diagnosing, treating conditions using these compounds, and methods of screening compounds that modulate FGF-CX polypeptide activity. Table A provides a summary of the FGF-CX nucleic acids and their encoded polypeptides.

TABLE A

| FGF-CXX | Internal | SEQ ID NO | SEQ ID NO | |
|---|---|---|---|---|
| Assignment | Identification | (nucleic acid) | (amino acid) | Homology |
| FGF-CX1a | CG53135-05 | 1 | 2 | Fibroblast growth factor-20 |
| | | | | (FGF-20) - Homo sapiens |
| FGF-CX1b | CG53135-01 | 3 | 4 | Fibroblast growth factor-20 |
| | | | | (FGF-20) - Homo sapiens |
| FGF-CX1c | CG53135-04 | 5 | 6 | Fibroblast growth factor-20 |
| | | | | (FGF-20) - Homo sapiens |
| FGF-CX1d | 250059596 | 7 | 8 | Fibroblast growth factor-20 |
| | | | | (FGF-20) - Homo sapiens |
| FGF-CX1e | 250059629 | 9 | 10 | Fibroblast growth factor-20 |
| | | | | (FGF-20) - Homo sapiens |
| FGF-CX1f | 250059669 | 11 | 12 | Fibroblast growth factor-20 |
| | | | | (FGF-20) - Homo sapiens |
| FGF-CX1g | 316351224 | 13 | 14 | Fibroblast growth factor-20 |
| | | | | (FGF-20) - Homo sapiens |
| FGF-CX1h | 317459553 | 15 | 16 | Fibroblast growth factor-20 |
| | | | | (FGF-20) - Homo sapiens |
| FGF-CX1i | 317459571 | 17 | 18 | Fibroblast growth factor-20 |
| | | | | (FGF-20) - Homo sapiens |
| FGF-CX1j | CG53135-02 | 19 | 20 | Fibroblast growth factor-20 |
| | | | | (FGF-20) - Homo sapiens |
| FGF-CX1k | CG53135-03 | 21 | 22 | Fibroblast growth factor-20 |
| | | | | (FGF-20) - Homo sapiens |
| FGF-CX1l | CG53135-06 | 23 | 24 | Fibroblast growth factor-20 |
| | | | | (FGF-20) - Homo sapiens |
| FGF-CX1m | CG53135-07 | 25 | 26 | Fibroblast growth factor-20 |
| | | | | (FGF-20) - Homo sapiens |
| FGF-CX1n | CG53135-08 | 27 | 28 | Fibroblast growth factor-20 |
| | | | | (FGF-20) - Homo sapiens |
| FGF-CX1o | CG53135-09 | 29 | 30 | Fibroblast growth factor-20 |
| | | | | (FGF-20) - Homo sapiens |
| FGF-CX1p | CG53135-10 | 31 | 32 | Fibroblast growth factor-20 |
| | | | | (FGF-20) - Homo sapiens |
| FGF-CX1q | CG53135-11 | 33 | 34 | Fibroblast growth factor-20 |
| | | | | (FGF-20) - Homo sapiens |
| FGF-CX1r | CG53135-12 | 35 | 36 | Fibroblast growth factor-20 |
| | | | | (FGF-20) - Homo sapiens |

As used herein, the terms “subject” and “subjects” refer to an animal, preferably a mammal, including a non-primate (e.g., a cow, pig, horse, cat, dog, rat, and mouse) and a primate (e.g., a monkey, such as a cynomolgous monkey, chimpanzee, and a human), and more preferably a human. In a certain embodiment, the subject is a mammal, preferably a human, who is suffering from a joint disease (e.g., osteoarthritis, other osteoarthritis-related disorders), ischemic stroke, hemorrhagic stroke, trauma, spinal cord damage, heavy metal or toxin poisoning, or neurodegenerative diseases (non-limiting examples being Alzheimer's, Parkinson's Disease, Amyotrophic Lateral Sclerosis, Huntington's Disease). In another embodiment, the subject is a mammal, preferably a human, who are at risk for a joint disease, ischemic stroke, hemorrhagic stroke, trauma, spinal cord damage, heavy metal or toxin poisoning, or neurodegenerative diseases. In one embodiment, the subject is a mammal, preferably a human, who is suffering from a joint disease, but who is not suffering from stroke or a neurodegenerative disease. The term “subject” is used interchangeably with “patient” in the present invention.

As used herein, the term “therapeutically effective amount” refers to the amount of a therapy (e.g., FGF-CX polypeptide), which is sufficient to reduce the severity of a disease (e.g., a joint disease, ischemic stroke, hemorrhagic stroke, trauma, spinal cord damage, heavy metal or toxin poisoning, or neurodegenerative diseases), reduce the duration of a disease, prevent the advancement of a disease, cause regression of a disease, ameliorate one or more symptoms associated with a disease, or enhance or improve the therapeutic effect(s) of another therapy.

Compositions comprising FGF-CX can also be administered in combination with one or more other therapies to prevent, treat, or ameliorate a disease (e.g., a joint disease, ischemic stroke, hemorrhagic stroke, trauma, spinal cord damage, heavy metal or toxin poisoning, or neurodegenerative diseases) or one or more symptoms thereof. In a preferred embodiment, compositions comprising FGF-CX is administered in combination with one or more other therapies known to be used in preventing, treating, or ameliorating a disease such as a joint disease, ischemic stroke, hemorrhagic stroke, trauma, spinal cord damage, heavy metal or toxin poisoning, or neurodegenerative diseases, or one or more symptoms thereof.

In one embodiment, during a combination therapy, FGF-CX polypeptide and/or another therapy are administered in a sub-optimal amount, e.g., an amount that does not manifest detectable therapeutic benefits when administered alone, as determined by methods known in the art. In such methods, co-administration of FGF-CX polypeptide and another therapy results in an overall improvement in effectiveness of treatment.

In one embodiment, FGF-CX polypeptide and one or more other therapies are administered within the same patient visit. In another embodiment, FGF-CX polypeptide is administered prior to the administration of one or more other therapies. In yet another embodiment, the FGF-CX polypeptide is administered subsequent to the administration of one or more other therapies. In a specific embodiment, FGF-CX polypeptide and one or more other therapies are cyclically administered to a subject. Cycling therapy involves the administration of FGF-CX polypeptide for a period of time, followed by the administration of one or more other therapies for a period of time and repeating this sequential administration. Cycling therapy can reduce the development of resistance to one or more of the therapies, avoid or reduce the side effects of one of the therapies, and/or improve the efficacy of the treatment.

Toxicity and therapeutic efficacy of a composition of the invention (e.g., FGF-CX polypeptide) can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio of LD₅₀/ED₅₀. Compositions that exhibit large therapeutic indices are preferred. While compositions that exhibit toxic side effects may be used, care should be taken to design a delivery system that targets such composition to the site of affected tissue in order to minimize potential damage to uninfected cells and, thereby, reduce side effects.

In one embodiment, the data obtained from the cell culture assays and animal studies can be used in formulating a range of dosage for use in humans. The dosage of complexes lies preferably within a range of circulating concentrations that include the ED₅₀ with little or no toxicity. The dosage may vary within this range depending upon the dosage form employed, the route of administration utilized, the severity of the disease, age and weight of the subject, and other factors normally considered by a medical professional (e.g., a physician). For any composition used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell cultures. Such information can be used to more accurately determine useful doses in humans. Levels in plasma may be measured, for example, by high performance liquid chromatography.

[Insert Prferred Dosage Ranges Here]

The appropriate and recommended dosages, formulation and routes of administration for treatment modalities such as chemotherapeutic agents, radiation therapy and biological/immunotherapeutic agents such as cytokines are known in the art and described in such literature as the Physician's Desk Reference (58th ed., 2004).

Various delivery systems are known and can be used to administer a composition of the invention. Such delivery systems include, but are not limited to, encapsulation in liposomes, microparticles, microcapsules, expression by recombinant cells, receptor-mediated endocytosis, construction of the nucleic acids of the invention as part of a retroviral or other vectors, etc. Methods of introduction include, but are not limited to, intradermal, intramuscular, intraperitoneal, intrathecal, intracerebroventricular, epidural, intravenous, subcutaneous, intranasal, intratumoral, transdermal, rectal, and oral routes. The compositions of the invention may be administered by any convenient route, for example, by infusion or bolus injection, by absorption through epithelial or mucocutaneous linings (e.g., oral mucosa, virginal mucosa, rectal and intestinal mucosa, etc.), and may be administered together with other biologically active agents. Administration can be systemic or local.

In some embodiments, it may be desirable to administer the pharmaceutical compositions of the invention locally to the area in need of treatment. This may be achieved by, for example, local infusion during surgery, or topical application, e.g., in conjunction with a wound dressing after surgery, by injection, by means of a catheter, by means of a suppository, or by means of an implant (said implant being of a porous, non-porous, or gelatinous material, including membranes, such as sialastic membranes, or fibers). In one embodiment, administration can be by direct injection at the site (or former site) of rapidly proliferating tissues which are most sensitive to an insult such radiation, chemotherapy, or chemical warfare agent.

In some embodiments, where the composition of the invention is a nucleic acid encoding a prophylactic or therapeutic agent, the nucleic acid can be administered in vivo to promote expression of their encoded proteins (e.g., FGF-CX polypeptide), by constructing the nucleic acid as part of an appropriate nucleic acid expression vector and administering it so that it becomes intracellular, e.g., by use of a retroviral vector, or by direct injection, or by use of microparticle bombardment (e.g., a gene gun), or coating with lipids or cell-surface receptors or transfecting agents, or by administering it in linkage to a homeobox-like peptide which is known to enter the nucleus, etc. Alternatively, a nucleic acid of the invention can be introduced intracellularly and incorporated within host cell DNA for expression, by homologous recombination.

The compositions of the invention include bulk drug compositions useful in the manufacture of pharmaceutical compositions that can be used in the preparation of unit dosage forms. In a preferred embodiment, a composition of the invention is a pharmaceutical composition. Such compositions comprise a prophylactically or therapeutically effective amount of one or more compositions (e.g., FGF-CX polypeptide) of the invention, and a pharmaceutically acceptable carrier. Preferably, the pharmaceutical compositions are formulated to be suitable for the route of administration to a subject.

In one embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the prophylactic or therapeutic agent is administered. Such pharmaceutical carriers can be sterile liquids, such as water and oils (e.g., oils of petroleum, animal, vegetable or synthetic origins, such as peanut oil, soybean oil, mineral oil, sesame oil and the like), or solid carriers, such as one or more substances which may also act as diluents, flavoring agents, solubilizers, lubricants, suspending agents, binders, tablet disintegrating agents, or encapsulating material. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include, but are not limited to, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, or a combination thereof. The composition, if desired, can also contain minor amounts of wetting or emulsifying agents, or pH buffering agents.

The compositions of the present invention may be formulated into any of many possible dosage forms such as, but not limited to, liquid syrups, tablets, capsules, gel capsules, soft gels, pills, powders, enemas, sustained-release formulations and the like. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. The composition can also be formulated as a suppository, with traditional binders and carriers such as triglycerides. Oral formulation can include standard carriers, such as pharmaceutical grades of mannitol, lactose, starch, magnesium stearate, sodium saccharine, cellulose, magnesium carbonate, etc.

In some embodiments, the compositions of the present invention may be formulated and used as foams. Pharmaceutical foams include formulations such as, but not limited to, emulsions, microemulsions, creams, jellies and liposomes. While basically similar in nature, these formulations vary in the components and the consistency of the final product. The preparation of such compositions and formulations is generally known to those skilled in the pharmaceutical and formulation arts and may be applied to the formulation of the compositions of the present invention.

A pharmaceutical composition of the invention is formulated to be compatible with its intended route of administration. In a specific embodiment, the composition is formulated in accordance with routine procedures as a pharmaceutical composition adapted for intravenous, subcutaneous, intramuscular, oral, intranasal, intratumoral or topical administration to human beings. Typically, compositions for intravenous administration are solutions in sterile isotonic aqueous buffer. Where necessary, the composition may also include a solubilizing agent and a local anesthetic such as lidocaine to ease pain at the site of the injection.

If the compositions of the invention are to be administered topically, the compositions can be formulated in the form of transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the polypeptides of the invention are in admixture with a topical delivery agent, such as but not limited to, lipids, liposomes, fatty acids, fatty acid esters, steroids, chelating agents and surfactants. Preferred lipids and liposomes include, but are not limited to, neutral (e.g. dioleoylphosphatidyl DOPE ethanolamine, dimyristoylphosphatidyl choline DMPC, distearolyphosphatidyl choline), negative (e.g. dimyristoylphosphatidyl glycerol DMPG), and cationic (e.g. dioleoyltetramethylaminopropyl DOTAP and dioleoylphosphatidyl ethanolamine DOTMA). The polypeptides of the invention may be encapsulated within liposomes or may form complexes thereto, in particular to cationic liposomes. Alternatively, the polypeptides may be complexed to lipids, in particular to cationic lipids. Preferred fatty acids and esters include, but are not limited to, arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcamitine, an acylcholine, or a C1-10 alkyl ester (e.g. isopropylmyristate IPM), monoglyceride, diglyceride, or pharmaceutically acceptable salt thereof. For non-sprayable topical dosage forms, viscous to semi-solid or solid forms comprising a carrier or one or more excipients compatible with topical application and having a dynamic viscosity preferably greater than water are typically employed. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well-known in the art.

If the method of the invention comprises intranasal administration of a composition, the composition can be formulated in an aerosol form, spray, mist or in the form of drops. In particular, prophylactic or therapeutic agents for use according to the present invention can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

If the method of the invention comprises oral administration, compositions can be formulated in the form of powders, granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

In one embodiment, the compositions of the invention are orally administered in conjunction with one or more penetration enhancers, e.g., surfactants and chelators. Preferred surfactants include, but are not limited to, fatty acids and esters or salts thereof, bile acids and salts thereof. In some embodiments, combinations of penetration enhancers are used, e.g., fatty acids/salts in combination with bile acids/salts. In a specific embodiment, sodium salt of lauric acid, capric acid is used in combination with UDCA. Further penetration enhancers include, but are not limited to, polyoxyethylene-9-lauryl ether, polyoxyethylene-20-cetyl ether. Compositions of the invention may be delivered orally in granular form including, but is not limited to, sprayed dried particles, or complexed to form micro or nanoparticles. Complexing agents that can be used for complexing with the peptide of the invention (e.g., FGF-CX polypeptide) include, but are not limited to, poly-amino acids, polyimines, polyacrylates, polyalkylacrylates, polyoxethanes, polyalkylcyanoacrylates, cationized gelatins, albumins, acrylates, polyethyleneglycols (PEG), polyalkylcyanoacrylates, DEAE-derivatized polyimines, pollulans, celluloses, and starches. Particularly preferred complexing agents include, but are not limited to, chitosan, N-trimethylchitosan, poly-L-lysine, polyhistidine, polyornithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG).

The method of the invention may comprise pulmonary administration, e.g., by use of an inhaler or nebulizer, of a composition formulated with an aerosolizing agent.

The method of the invention may comprise administration of a composition formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container, such as an ampoule or sachette, indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include, but are not limited to, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Non-limiting examples of pharmaceutically acceptable salts are acetate, benzenesulfonate, benzoate, bicarbonate, bitartrate, bromide, calcium acetate, camsylate, carbonate, chloride, citrate, dihydrochloride, edetate, edisylate, estolate, esylate, fumarate, glucaptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate mesylate, methylbromide, methylnitrate, methylsulfate, mucate, napsylate, nitrate, pamoate (embonate), pantothenate, phosphateldiphosphate, polygalacturonate, salicylate, stearate, subacetate, succinate, sulfate, tannate, tartrate, teoclate, triethiodide, benzathine, chloroprocaine, choline, diethanolamine, ethylenediamine, meglumine, procaine, aluminium, calcium, lithium, magnesium, potassium, sodium, and zinc.

In addition to the formulations described previously, the compositions may also be formulated as a depot preparation. Such long acting formulations may be administered by implantation (for example, subcutaneously or intramuscularly) or by intramuscular injection. Thus, for example, the compositions may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

In one embodiment, the ingredients of the compositions of the invention (e.g., FGF-CX) are derived from a subject that is the same species origin or species reactivity as recipient of such compositions.

The invention also provides kits for carrying out the therapeutic regimens of the invention. Such kits comprise in one or more containers prophylactically or therapeutically effective amounts of the composition of the invention (e.g., FGF-CX polypeptide) in pharmaceutically acceptable form. The composition in a vial of a kit of the invention may be in the form of a pharmaceutically acceptable solution, e.g., in combination with sterile saline, dextrose solution, or buffered solution, or other pharmaceutically acceptable sterile fluid. Alternatively, the composition may be lyophilized or desiccated; in this instance, the kit optionally further comprises in a container a pharmaceutically acceptable solution (e.g., saline, dextrose solution, etc.), preferably sterile, to reconstitute the composition to form a solution for injection purposes.

In another embodiment, a kit of the invention further comprises a needle or syringe, preferably packaged in sterile form, for injecting the formulation, and/or a packaged alcohol pad. Instructions are optionally included for administration of the formulations of the invention by a clinician or by the patient.

In some embodiments, the present invention provides kits comprising a plurality of containers each comprising a pharmaceutical formulation or composition comprising a dose of the composition of the invention (e.g., FGF-CX polypeptide) sufficient for a single administration.

As with any pharmaceutical product, the packaging material and container are designed to protect the stability of the product during storage and shipment. In one embodiment, compositions of the invention are stored in containers with biocompatible detergents, including but not limited to, lecithin, taurocholic acid, and cholesterol; or with other proteins, including but not limited to, gamma globulins and serum albumins. Further, the products of the invention include instructions for use or other informational material that advise the physician, technician, or patient on how to appropriately prevent or treat the disease or disorder in question.

The present invention is further illustrated by the following Examples.

### 5.1. EXAMPLE 1

### Sequence Analysis

Details of the sequence relatedness and domain analysis for each FGF-CX are presented in Table 1A. The FGF-CX1 clone was analyzed, and the nucleotide and encoded polypeptide sequences are shown in Table 1A.

TABLE 1A

FGF-CX1 Sequence Analysis

| | | |
|---|---|---|
| FGF-CX1a, CG53135-05 | SEQ ID NO: 1 | 636 bp |
| DNA Sequence | ORF Start: ATG at 1 | ORF Stop: end of sequence |

ATGGCTCCGCTGGCTGAAGTTGGTGGTTTCCTGGGCGGTCTGGAGGGTCTGGGTCAGCAGGTTGGTTC
TCACTTCCTGCTGCCGCCGGCTGGTGAACGTCCGCCACTGCTGGGTGAACGTCGCTCCGCAGCTGAAC
GCTCCGCTCGTGGTGGCCCGGGTGCTGCTCAGCTGGCTCACCTGCATGGTATCCTGCGTCGCCGTCAG
CTGTACTGCCGTACTGGTTTCCACCTGCAGATCCTGCCGGATGGTTCTGTTCAGGGTACCCGTCAGGA
CCACTCTCTGTTCGGTATCCTGGAATTCATCTCTGTTGCTGTTGGTCTGGTTTCTATCCGTGGTGTTG
ACTCTGGCCTGTACCTGGGTATGAACGACAAAGGCGAACTGTACGGTTCTGAAAAACTGACCTCTGAA
TGCATCTTCCGTGAACAGTTTGAAGAGAACTGGTACAACACCTACTCTTCCAACATCTACAAACATGG
TGACACCGGCCGTCGCTACTTCGTTGCTCTGAACAAAGACGGTACCCCGCGTGATGGTGCTCGTTCTA
AACGTCACCAGAAATTCACCCACTTCCTGCCGCGCCCAGTTGACCCGGAGCGTGTTCCAGAACTGTAT
AAAGACCTGCTGATGTACACCTAA

| | | | |
|---|---|---|---|
| FGF-CX1a, CG53135-05 | SEQ ID NO: 2 | 211 aa | MW at 23498.4kD |
| Protein Sequence | | | |

MAPLAEVGGFLGGLEGLGQQVGSHFLLPPAGERPPLLGERRSAAERSARGGPGAAQLAHLHGILRRRQ
LYCRTGFHLQILPDGSVQGTRQDHSLFGILEFISVAVGLVSIRGVDSGLYLGMNDKGELYGSEKLTSE
CIFREQFEENWYNTYSSNIYKHGDTGRRYFVALNKDGTPRDGARSKRHQKFTHFLPRPVDPERVPELY
KDLLMYT

| | | |
|---|---|---|
| FGF-CX1b, CG53135-01 | SEQ ID NO: 3 | 633 bp |
| DNA Sequence | ORF Start: ATG at 1 | ORF Stop: |

ATGGCTCCCTTAGCCGAAGTCGGGGGCTTTCTGGGCGGCCTGGAGGGCTTGGGCCAGCAGGTGGGTTC
GCATTTCCTGTTGCCTCCTGCCGGGGAGCGGCCGCCGCTGCTGGGCGAGCGCAGGAGCGCGGCGGAGC
GGAGCGCGCGCGGCGGGCCGGGGGCTGCGCAGCTGGCGCACCTGCACGGCATCCTGCGCCGCCGGCAG
CTCTATTGCCGCACCGGCTTCCACCTGCAGATCCTGCCCGACGGCAGCGTGCAGGGCACCCGGCAGGA
CCACAGCCTCTTCGGTATCTTGGAATTCATCAGTGTGGCAGTGGGACTGGTCAGTATTAGAGGTGTGG
ACAGTGGTCTCTATCTTGGAATGAATGACAAAGGAGAACTCTATGGATCAGAGAAACTTACTTCCGAA
TGCATCTTTAGGGAGCAGTTTGAAGAGAACTGGTATAACACCTATTCATCTAACATATATAAACATGG
AGACACTGGCCGCAGGTATTTTGTGGCACTTAACAAAGACGGAACTCCAAGAGATGGCGCCAGGTCCA
AGAGGCATCAGAAATTTACACATTTCTTACCTAGACCAGTGGATCCAGAAAGAGTTCCAGAATTGTAC
AAGGACCTACTGATGTACACT

| | | | |
|---|---|---|---|
| FGF-CX1b, CG53135-01 | SEQ ID NO: 4 | 211 aa | MW at 23498.4kD |
| Protein Sequence | | | |

MAPLAEVGGFLGGLEGLGQQVGSHFLLPPAGERPPLLGERRSAAERSARGGPGAAQLAHLHGILRRRQ
LYCRTGFHLQILPDGSVQGTRQDHSLFGILEFISVAVGLVSIRGVDSGLYLGMNDKGELYGSEKLTSE
CIFREQFEENWYNTYSSNIYKHGDTGRRYFVALNKDGTPRDGARSKRHQKFTHFLPRPVDPERVPELY
KDLLMYT

| | | |
|---|---|---|
| FGF-CX1c, CG53135-04 | SEQ ID NO: 5 | 540 bp |
| DNA Sequence | ORF Start: ATG at 1 | ORE Stop: end of sequence |

ATGGCTCCCTTAGCCGAAGTCGGGGGCTTTCTGGGCGGCCTGGAGGGCTTGGGCCAGCCGGGGGCAGC
GCAGCTGGCGCACCTGCACGGCATCCTGCGCCGCCGGCAGCTCTATTGCCGCACCGGCTTCCACCTGC
AGATCCTGCCCGACGGCAGCGCGCAGGGCACCCGGCAGGACCACAGCCTCTTCGGTATCTTGGAATTC
ATCAGTGTGGCAGTGGGACTGGTCAGTATTAGAGGTGTGGACAGTGGTCTCTATCTTGGAATGAATGA
CAAAGGAGAACTCTATGGATCAGAGAAACTTACTTCCGAATGCATCTTTAGGGAGCAGTTTGAAGAGA
ACTGGTATAACACCTATTCATCTAACATATATAAACATGGAGACACTGGCCGCAGGTATTTTGTGGCA
CTTAACAAAGACGGAACTCCAAGAGATGGCGCCAGGTCCAAGAGGCATCAGAAATTTACACATTTCTT
ACCTAGACCAGTGGATCCAGAAAGAGTTCCAGAATTGTACAAGGACCTACTGATGTACACTTAG

| | | | |
|---|---|---|---|
| FGF-CX1c, CG53135-04 | SEQ ID NO: 6 | 179 aa | MW at 20118.6kD |
| Protein Sequence | | | |

MAPLAEVGGFLGGLEGLGQPGAAQLAHLHGILRRRQLYCRTGFHLQILPDGSAQGTRQDHSLFGILEF
ISVAVGLVSIRGVDSGLYLGMNDKGELYGSEKLTSECIFREQFEENWYNTYSSNIYKHGDTGRRYFVA
LNKDGTPRDGARSKRHQKFTHFLPRPVDPERVPELYKDLLMYT

| | | |
|---|---|---|
| FGF-CX1d, 250059596 | SEQ ID NO: 7 | 556 bp |
| DNA Sequence | ORE Start: | ORE Stop: |

CACCAGATCTATGGCTCCCTTAGCCGAAGTCGGGGGCTTTCTGGGCGGCCTGGAGGGCTTGGGCCAGC
CGGGGGCAGCGCAGCTGGCGCACCTGCACGGCATCCTGCGCCGCCGGCAGCTCTATTGCCGCACCGGC
TTCCACCTGCAGATCCTGCCCGACGGCAGCGTGCAGGGCACCCGGCAGGACCACAGCCTCTTCGGTAT
CTTGGAATTCATCAGTGTGGCAGTGGGACTGGTCAGTATTAGAGGTGTGGACAGTGGTCTCTATCTTG
GAATGAATGACAAAGGAGAACTCTATGGATCAGAGAAACTTACTTCCGAATGCATCTTTAGGGAGCAG
TTTGAAGAGAACTGGTATAACACCTATTCATCTAACATATATAAACATGGAGACACTGGCCGCAGGTA
TTTTGTGGCACTTAACAAAGACGGAACTCCAAGAGATGGCGCCAGGTCCAAGAGGCATCAGAAATTTA
CACATTTCTTACCTAGACCAGTGGATCCAGAAAGAGTTCCAGAATTGTACAAGGACCTACTGATGTAC
ACTGTCGACGGC

| | | | |
|---|---|---|---|
| FGF-CX1d, 250059596 | SEQ ID NO: 8 | 185 aa | MW at 20762.3kD |
| Protein Sequence | | | |

TRSMAPLAEVGGFLGGLEGLGQPGAAQLAHLHGILRRRQLYCRTGFHLQILPDGSVQGTRQDHSLFGI
LEFISVAVGLVSIRGVDSGLYLGMNDKGELYGSEKLTSECIFREQFEENWYNTYSSNIYKHGDTGRRY
FVALNKDGTPRDGARSKRHQKFTHFLPRPVDPERVPELYKDLLMYTVDG

| | | |
|---|---|---|
| FGF-CX1e, 250059629 | SEQ ID NO: 9 | 415 bp |
| DNA Sequence | ORE Start: | ORE Stop: |

CACCAGATCTATCCTGCGCCGCCGGCAGCTCTATTGCCGCACCGGCTTCCACCTGCAGATCCTGCCCG
ACGGCAGCGTGCAGGGCACCCGGCAGGACCACAGCCTCTTCGGTATCTTGGAATTCATCAGTGTGGCA
GTGGGACTGGTCAGTATTAGAGGTGTGGACAGTGGTCTCTATCTTGGAATGAATGACAAAGGAGAACT
CTATGGATCAGAGAAACTTACTTCCGAATGCATCTTTAGGGAGCAGTTTGAAGAGAACTGGTATAACA
CCTATTCATCTAACATATATAAACATGGAGACACTGGCCGCAGGTATTTTGTGGCACTTAACAAAGAC
GGAACTCCAAGAGATGGCGCCAGGTCCAAGAGGCATCAGAAATTTACACATTTCTTACCTAGACCAGT
CGACGGC

| | | | |
|---|---|---|---|
| FGF-CX1e, 250059629 | SEQ ID NO: 10 | 138 aa | MW at 15847.7kD |
| Protein Sequence | | | |

TRSILRRRQLYCRTGFHLQILPDGSVQGTRQDHSLFGILEFISVAVGLVSIRGVDSGLYLGMNDKGEL
YGSEKLTSECIFREQFEENWYNTYSSNIYKHGDTGRRYFVALNKDGTPRDGARSKRRQKFTHFLPRPV
DG

| | | |
|---|---|---|
| FGF-CX1f, 250059669 | SEQ ID NO: 11 | 466 bp |
| DNA Sequence | ORE Start: | ORE Stop: |

CACCAGATCTATCCTGCGCCGCCGGCAGCTCTATTGCCGCACCGGCTTCCACCTGCAGATCCTGCCCG
ACGGCAGCGTGCAGGGCACCCGGCAGGACCACAGCCTCTTCGGTATCTTGGAATTCATCAGTGTGGCA
GTGGGACTGGTCAGTATTAGAGGTGTGGACAGTGGTCTCTATCTTGGAATGAATGACAAAGGAGAACT
CTATGGATCAGAGAAACTTACTTCCGAATGCATCTTTAGGGAGCAGTTTGAAGAGAACTGGTATAACA
CCTATTCATCTAACATATATAAACATGGAGACACTGGCCGCAGGTATTTTGTGGCACTTAACAAAGAC
GGAACTCCAAGAGATGGCGCCAGGTCCAAGAGGCATCAGAAATTTACACATTTCTTACCTAGACCAGT
GGATCCAGAAAGAGTTCCAGAATTGTACAAGGACCTACTGATGTACACTGTCGACGGC

| | | | |
|---|---|---|---|
| FGF-CX1f, 250059669 | SEQ ID NO: 12 | 155 aa | MW at 17911.1kD |
| Protein Sequence | | | |

TRSILRRRQLYCRTGFHLQILPDGSVQGTRQDHSLFGILEFISVAVGLVSIRGVDSGLYLGMNDKGEL
YGSEKLTSECIFREQFEENWYNTYSSNIYKHGDTGRRYFVALNKDGTPRDGARSKRHQKFTHFLPRPV
DPERVPELYKDLLMYTVDG

| | | |
|---|---|---|
| FGF-CX1g, 316351224 | SEQ ID NO: 13 | 549 bp |
| DNA Sequence | ORF Start: | ORF Stop: |

AGATCTATGGCTCCCTTAGCCGAAGTCGGGGGCTTTCTGGGCGGCCTGGAGGGCTTGGGCCAGCCGGG
GGCAGCGCAGCTGGCGCACCTGCACGGCATCCTGCGCCGCCGGCAGCTCTATTGCCGCACCGGCTTCC
ACCTGCAGATCCTGCCCGACGGCAGCGTGCAGGGCACCCGGCAGGACCACAGCCTCTTCGGTATCTTG
GAATTCATCAGTGTGGCAGTGGGACTGGTCAGTATTAGAGGTGTGGACAGTGGTCTCTATCTTGGAAT
GAATGACAAAGGAGAACTCTATGGATCAGAGAAACTTACTTCCGAATGCATCTTTAGGGAGCAGTTTG
AAGAGAACTGGTATAACACCTATTCATCTAACATATATAAACATGGAGACACTGGCCGCAGGTATTTT
GTGGCACTTAACAAAGACGGAACTCCAAGAGATGGCGCCAGGTCCAAGAGGCATCAGAAATTTACACA
TTTCTTACCTAGACCAGTGGATCCAGAAAGAGTTCCAGAATTGTACAAGGACCTACTGATGTACACTC
TCGAG

| | | | |
|---|---|---|---|
| FGF-CX1g, 316351224 | SEQ ID NO: 14 | 183 aa | MW at 20632.2kD |
| Protein Sequence | | | |

RSMAPLAEVGGFLGGLEGLGQPGAAQLAHLHGILRRRQLYCRTGFHLQILPDGSVQGTRQDHSLFGIL
EFISVAVGLVSIRGVDSGLYLGMNDKGELYGSEKLTSECIFREQFEENWYNTYSSNIYKHGDTGRRYF
VALNKDGTPRDGARSKRHQKFTHFLPRPVDPERVPELYKDLLMYTLE

| | | |
|---|---|---|
| FGF-CX1h, 317459553 | SEQ ID NO: 15 | 408 bp |
| DNA Sequence | ORE Start: | ORE Stop: |

AGATCTATCCTGCGCCGCCGGCAGCTCTATTGCCGCACCGGCTTCCACCTGCAGATCCTGCCCGACGG
CAGCGTGCAGGGCACCCGGCAGGACCACAGCCTCTTCGGTATCTTGGAATTCATCAGTGTGGCAGTGG
GACTGGTCAGTATTAGAGGTGTGGACAGTGGTCTCTATCTTGGAATGAATGACAAAGGAGAACTCTAT
GGATCAGAGAAACTTACTTCCGAATGCATCTTTAGGGAGCAGTTTGAAGAGAACTGGTATAACACCTA
TTCATCTAACATATATAAACATGAAGACACTGGCCGCAGGTATTTTGTGGCACTTAACAAAGACGGAA
CTCCAAGAGATGGCGCCAGGTCCAAGAGGCATCAGAAATTTACACATTTCTTACCTAGACCACTCGAG

| | | | |
|---|---|---|---|
| FGF-CX1h, 317459553 | SEQ ID NO: 16 | 136 aa | MW at 15789.6kD |
| Protein Sequence | | | |

RSILRRRQLYCRTGFHLQILPDGSVQGTRQDHSLFGILEFISVAVGLVSIRGVDSGLYLGMNDKGELY
GSEKLTSECIFREQFEENWYNTYSSNIYKHEDTGRRYFVALNKDGTPRDGARSKRHQKFTHFLPRPLE

| | | |
|---|---|---|
| FGF-CX1i, 317459571 | SEQ ID NO: 17 | 408 bp |
| DNA Sequence | ORE Start: 1 | ORE Stop: |

AGATCTATCCTGCGCCGCCGGCAGCTCTATTGCCGCACCGGCTTCCACCTGCAGATCCTGCCCGACGG
CAGCGTGCAGGGCACCCGGCAGGACCACAGCCTCTTCGGTATCTTGGAATTCATCAGTGTGGCAGTGG
GACTGGTCAGTATTAGAGGTGTGGACAGTGGTCTCTATCTTGGAATGAATGACAAAGGAGAACTCTAT
GGATCAGAGAAACTTACTTCCGAATGCATCTTTAGGGAGCAGTTTGAAGAGAACTGGTATAACACCTA
TTCATCTAACATATATAAACATGGAGACACTGGCCGCAGGTATTTTGTGGCACTTAACAAAGACGGAA
CTCCAAGAGATGGCGCCAGGTCCAAGAGGCATCAGAAATTTACACATTTCTTACCTAGACCACTCGAG

| | | | |
|---|---|---|---|
| FGF-CX1i, 317459571 | SEQ ID NO: 18 | 136 aa | MW at 15717.6kD |
| Protein Sequence | | | |

RSILRRRQLYCRTGFHLQILPDGSVQGTRQDHSLFGILEFISVAVGLVSIRGVDSGLYLGMNDKGELY
GSEKLTSECIFREQFEENWYNTYSSNIYKHGDTGRRYFVALNKDGTPRDGARSKRRQKFTHFLPRPLE

| | | |
|---|---|---|
| FGF-CX1j, CG53135-02 | SEQ ID NO: 19 | 477 bp |
| DNA Sequence | ORE Start: ATG at 1 | ORE Stop: end of sequence |

ATGGCTCAGCTGGCTCACCTGCATGGTATCCTCCCTCGCGGTCAGCTGTACTCCCGTACTGGTTTCCA
CCTGCAGATCCTGCCGGATGGTTCTGTTCAGGGTACCCGTCAGGACCACTCTCTGTTCGGTATCCTGG
AATTCATCTCTGTTGCTGTTCCTCTGGTTTCTATCCGTGGTGTTGACTCTGGCCTGTACCTGGGTATG
AACGACAAAGGCGAACTGTACGGTTCTGAAAAACTGACCTCTGAATGCATCTTCCGTGAACAGTTTGA
AGAGAACTGGTACAACACCTACTCTTCCAACATCTACAAACATGGTGACACCGGCCGTCGCTACTTCG
TTGCTCTGAACAAAGACGGTACCCCGCGTGATGGTGCTCGTTCTAAACGTCACCAGAAATTCACCCAC
TTCCTGCCGCGCCCAGTTGACCCGGAGCGTGTTCCAGAACTGTATAAAGACCTGCTGATGTACACCTA
A

| | | | |
|---|---|---|---|
| FGF-CX1j, CG53135-02 | SEQ ID NO: 20 | 158 aa | MW at 18254.6kD |
| Protein Sequence | | | |

MAQLAHLHGILRRRQLYCRTGFHLQILPDGSVQGTRQDHSLFGILEFISVAVGLVSIRGVDSGLYLGM
NDKGELYGSEKLTSECIFREQFEENWYNTYSSNIYKHGDTGRRYFVALNKDGTPRDGARSKRHQKFTH
FLPRPVDPERVPELYKDLLMYT

| | | |
|---|---|---|
| FGF-CX1k, CG53135-03 | SEQ ID NO: 21 | 636 bp |
| DNA Sequence | ORE Start: ATG at 1 | ORF Stop: end of sequence |

ATGGCTCCCTTAGCCGAAGTCGGGGGCTTTCTGGGCGGCCTGGAGGGCTTGGGCCAGCAGGTGGGTTC
GCATTTCCTGTTGCCTCCTGCCGGGGAGCGGCCGCCGCTGCTGGGCGAGCGCAGGAGCGCGGCGGAGC
GGAGCGCGCGCGGCGGGCCGGGGGCTGCGCAGCTGGCGCACCTGCACGGCATCCTGCGCCGCCGGCAG
CTCTATTGCCGCACCGGCTTCCACCTGCAGATCCTGCCCGACGGCAGCGTGCAGGGCACCCGGCAGGA
CCACAGCCTCTTCGGTATCTTGGAATTCATCAGTGTGGCAGTGGGACTGGTCAGTATTAGAGGTGTGG
ACAGTGGTCTCTATCTTGGAATGAATGACAAAGGAGAACTCTATGGATCAGAGAAACTTACTTCCGAA
TGCATCTTTAGGGAGCAGTTTGAAGAGAACTGGTATAACACCTATTCATCTAACATATATAAACATGG
AGACACTGGCCGCAGGTATTTTGTGGCACTTAACAAAGACGGAACTCCAAGAGATGGCGCCAGGTCCA
AGAGGCATCAGAAATTTACACATTTCTTACCTAGACCAGTGGATCCAGAAAGAGTTCCAGAATTGTAC
AAGGACCTACTGATGTACACTTGA

| | | | |
|---|---|---|---|
| FGF-CX1k, CG53135-03 | SEQ ID NO: 22 | 211 aa | MW at 23498.4kD |
| Protein Sequence | | | |

MAPLAEVGGFLGGLEGLGQQVGSHFLLPPAGERPPLLGERRSAAERSARGGPGAAQLAHLHGILRRRQ
LYCRTGFHLQILPDGSVQGTRQDHSLFGILEFISVAVGLVSIRGVDSGLYLGMNDKGELYGSEKLTSE
CIFREQFEENTYNTYSSNIYKHGDTGRRYFVALNKDGTPRDGARSKRHQKFTHFLPRPVDPERVPELY
KDLLMYT

| | | |
|---|---|---|
| FGF-CX1l, CG53135-06 | SEQ ID NO: 23 | 540 bp |
| DNA Sequence | ORE Start: ATG at 1 | ORE Stop: end of sequence |

ATGGCTCCCTTAGCCGAAGTCGGGGGCTTTCTGGGCGGCCTGGAGGGCTTGGGCCAGCCGGGGGCAGC
GCAGCTGGCGCACCTGCACGGCATCCTGCGCCGCCGGCAGCTCTATTGCCGCACCGGCTTCCACCTGC
AGATCCTGCCCGACGGCAGCGTGCAGGGCACCCGGCAGGACCACAGCCTCTTCGGTATCTTGGAATTC
ATCAGTGTGGCAGTGGGACTGGTCAGTATTAGAGGTGTGGACAGTGGTCTCTATCTTGGAATGAATGA
CAAAGGAGAACTCTATGGATCAGAGAAACTTACTTCCGAATGCATCTTTAGGGAGCAGTTTGAAGAGA
ACTGGTATAACACCTATTCATCTAACATATATAAACATGGAGACACTGGCCGCAGGTATTTTGTGGCA
CTTAACAAAGACGGAACTCCAAGAGATGGCGCCAGGTCCAAGAGGCATCAGAAATTTACACATTTCTT
ACCTAGACCAGTGGATCCAGAAAGAGTTCCAGAATTGTACAAGGACCTACTGATGTACACTTAG

| | | | |
|---|---|---|---|
| FGF-CX1l, CG53135-06 | SEQ ID NO: 24 | 179 aa | MW at 20146.7kD |
| Protein Sequence | | | |

MAPLAEVGGFLGGLEGLGQPGAAQLAHLHGILRRRQLYCRTGFHLQILPDGSVQGTRQDHSLFGILEF
ISVAVGLVSIRGVDSGLYLGMNDKGELYGSEKLTSECIFREQFEENWYNTYSSNIYKHGDTGRRYFVA
LNKDGTPRDGARSKRHQKFTHFLPRPVDPERVPELYKDLLMYT

| | | |
|---|---|---|
| FGF-CX1m, CG53135-07 | SEQ ID NO: 25 | 54 bp |
| DNA Sequence | ORE Start: ATG at 1 | ORE Stop: |

ATGGCTCCCTTAGCCGAAGTCGGGGGCTTTCTGGGCGGCCTGGAGGGCTTGGGC

| | | | |
|---|---|---|---|
| FGF-CX1m, CG53135-07 | SEQ ID NO: 26 | 18 aa | MW at 1688.0kD |
| Protein Sequence | | | |

MAPLAEVGGFLGGLEGLG

| | | |
|---|---|---|
| FGF-CX1n, CG53135-08 | SEQ ID NO: 27 | 63 bp |
| DNA Sequence | ORF Start: | ORF Stop: |

GAGCGGCCGCCGCTGCTGGGCGAGCGCAGGAGCGCGGCGGAGCGGAGCGCGCGCGGCGGGCCG

| | | | |
|---|---|---|---|
| FGF-CX1n, CG53135-08 | SEQ ID NO: 28 | 21 aa | MW at 2262.5kD |
| Protein Sequence | | | |

ERPPLLGERRSAAERSARGGP

| | | |
|---|---|---|
| FGF-CX1o, CG53135-09 | SEQ ID NO: 29 | 63 bp |
| DNA Sequence | ORF Start: | ORF Stop: |

CGCAGGTATTTTGTGGCACTTAACAAAGACGGAACTCCAAGAGATGGCGCCAGGTCCAAGAGG

| | | | |
|---|---|---|---|
| FGF-CX1o, CG53135-09 | SEQ ID NO: 30 | 21 aa | MW at 2463.8kD |
| Protein Sequence | | | |

RRYFVALNKDGTPRDGARSKR

| | | |
|---|---|---|
| FGF-CX1p, CG53135-10 | SEQ ID NO: 31 | 60 bp |
| DNA Sequence | ORE Start: | ORF Stop: |

CCTAGACCAGTGGATCCAGAAAGAGTTCCAGAATTGTACAAGGACCTACTGATGTACACT

| | | | |
|---|---|---|---|
| FGF-CX1p, CG53135-10 | SEQ ID NO: 32 | 20 aa | MW at 2431.8kD |
| Protein Sequence | | | |

PRPVDPERVPELYKDLLMYT

| | | |
|---|---|---|
| FGF-CX1q, CG53135-11 | SEQ ID NO: 33 | 51 bp |
| DNA Sequence | ORF Start: ATG at 1 | ORE Stop: |

ATGAACGACAAGGGCGAGCTGTACGGCAGCGAGAAGCTGACCAGCGAGTGC

| | | | |
|---|---|---|---|
| FGF-CX1q, CG53135-11 | SEQ ID NO: 34 | 17 aa | MW at 1904.1kD |
| Protein Sequence | | | |

MNDKGELYGSEKLTSEC

| | | |
|---|---|---|
| FGF-CX1r, CG53135-12 | SEQ ID NO: 35 | 633 bp |
| DNA Sequence | ORF Start: ATG at 1 | ORF Stop: |

ATGGCTCCCTTAGCCGAAGTCGGGGGCTTTCTGGGCGGCCTGGAGGGCTTGGGCCAGCAGGTGGGTTC
GCATTTCCTGTTGCCTCCTGCCGGGGAGCGGCCGCCGCTGCTGGGCGAGCGCAGGAGCGCGGCGGAGC
GGAGCGCGCGCGGCGGGCCGGGGGCTGCGCAGCTGGCGCACCTGCACGGCATCCTGCGCCGCCGGCAG
CTCTATTGCCGCACCGGCTTCCACCTGCAGATCCTGCCCGACGGCAGCGTGCAGGGCACCCGGCAGGA
CCACAGCCTCTTCGGTATCTTGGAATTCATCAGTGTGGCAGTGGGACTGGTCAGTATTAGAGGTGTGG
ACAGTGGTCTCTATCTTGGAATGAATGACAAAGGAGAACTCTATGGATCAGAGAAACTTACTTCCGAA
TGCATCTTTAGGGAGCAGTTTGAAGAGAACTGGTATAACACCTATTCATCTAACATATATAAACATGG
AGACACTGGCCGCAGGTATTTTGTGGCACTTAACAAAGACGGAACTCCAAGAGATGGCGCCAGGTCCA
AGAGGCATCAGAAATTTACACATTTCTTACCTAGACCAGTGGATCCAGAAAGAGTTCCAGAATTGTAC
AAGAACCTACTGATGTACACT

| | | | |
|---|---|---|---|
| FGF-CX1r, CG53135-12 | SEQ ID NO: 36 | 211 aa | MW at 23497.4kD |
| Protein Sequence | | | |

MAPLAEVGGFLGGLEGLGQQVGSHFLLPPAGERPPLLGERRSAAERSARGGPGAAQLAHLHGILRRRQ
LYCRTGFHLQILPDGSVQGTRQDHSLFGILEFISVAVGLVSIRGVDSGLYLGMNDKGELYGSEKLTSE
CIFREQFEENWYNTYSSNIYKHGDTGRRYFVALNKDGTPRDGARSKRHQKFTHFLPRPVDPERVPELY
KNLLMYT

A ClustalW comparison of the above protein sequences yields the following sequence alignment shown in Table 1B.

TABLE 1B

Comparison of the FGF-CX1 protein sequences.

| | |
|---|---|
| FGF-CX1a | MAPLAEVGGFLGGLEGLGQQVGSHFLLPPAGERPPLLGERRSAAERSARGGPGAAQLAHL |
| FGF-CX1b | MAPLAEVGGFLGGLEGLGQQVGSHFLLPPAGERPPLLGERRSAAERSARGGPGAAQLAHL |
| FGF-CX1c | ------------------------------------------------------------ |
| FGF-CX1d | ------------------------------------------------------------ |
| FGF-CX1e | -----------------------------------------------------------T |
| FGF-CX1f | -------------------------------TRSILRRRQLYCRTGFHLQILPDGSVQGT |
| FGF-CX1g | ------------------------------------------------------------ |
| FGF-CX1h | ------------------------------------------------------------ |
| FGF-CX1i | ------------------------------------------------------------ |
| FGF-CX1j | -------------------------MAQLAHLHGILRRRQLYCRTGFHLQILPDGSVQGT |
| FGF-CX1k | MAPLAEVGGFLGGLEGLGQQVGSHFLLPPAGERPPLLGERRSAAERSARGGPGAAQLAHL |
| FGF-CX1l | ----MAPLAEVGGFLGGLEGLGQPGAAQLAHLHGILRRRQLYCRTGFHLQILPDGSVQGT |
| FGF-CX1m | ------------------------------------------------------------ |
| FGF-CX1n | ------------------------------------------------------------ |
| FGF-CX1o | ------------------------------------------------------------ |
| FGF-CX1p | ------------------------------------------------------------ |
| FGF-CX1q | ------------------------------------------------------------ |
| FGF-CX1r | MAPLAEVGGFLGGLEGLGQQVGSHFLLPPAGERPPLLGERRSAAERSARGGPGAAQLAHL |
| FGF-CX1a | HGILRRRQLYCRTGFHLQILPDGSVQGTRQDHSLFGILEFISVAVGLVSIRGVDSGLYLG |
| FGF-CX1b | HGILRRRQLYCRTGFHLQILPDGSVQGTRQDHSLFGILEFISVAVGLVSIRGVDSGLYLG |
| FGF-CX1c | ------------------------------------------------------------ |
| FGF-CX1d | ------------------------------------------------------------ |
| FGF-CX1e | RSILRRRQLYCRTGFHLQILPDGSVQGTRQDHSLFGILEFISVAVGLVSIRGVDSGLYLG |
| FGF-CX1f | RQDHSLFGILEFISVAVGLVSIRGVDSGLYLGMNDKGELYGSEKLTSECIFREQFEENWY |
| FGF-CX1g | ------------------------------------------------------------ |
| FGF-CX1h | RSILRRRQLYCRTGFHLQILPDGSVQGTRQDHSLFGILEFISVAVGLVSIRGVDSGLYLG |
| FGF-CX1i | RSILRRRQLYCRTGFHLQILPDGSVQGTRQDHSLFGILEFISVAVGLVSIRGVDSGLYLG |
| FGF-CX1j | RQDHSLFGILEFISVAVGLVSIRGVDSGLYLGMNDKGELYGSEKLTSECIFREQFEENWY |
| FGF-CX1k | HGILRRRQLYCRTGFHLQILPDGSVQGTRQDHSLFGILEFISVAVGLVSIRGVDSGLYLG |
| FGF-CX1l | RQDHSLFGILEFISVAVGLVSIRGVDSGLYLGMNDKGELYGSEKLTSECIFREQFEENWY |
| FGF-CX1m | ------------------------------------------------------------ |
| FGF-CX1n | ------------------------------------------------------------ |
| FGF-CX1o | ------------------------------------------------------------ |
| FGF-CX1p | ------------------------------------------------------------ |
| FGF-CX1q | ------------------------------------------------------------ |
| FGF-CX1r | HGILRRRQLYCRTGFHLQILPDGSVQGTRQDHSLFGILEFISVAVGLVSIRGVDSGLYLG |
| FGF-CX1a | MNDKGELYGSEKLTSECIFREQFEENWYNTYSSNIYKHGDTGRRYFVALNKDGTPRDGAR |
| FGF-CX1b | MNDKGELYGSEKLTSECIFREQFEEMWYNTYSSNIYKHGDTGRRYFVALNKDGTPRDGAR |
| FGF-CX1c | ---------------------------------------------MAPLAEVGGFLGGLE |
| FGF-CX1d | ------------------------------------------TRSMAPLAEVGGFLGGLE |
| FGF-CX1e | MNDKGELYGSEKLTSECIFREQFEENWYNTYSSNIYKHGDTGRRYFVALNKDGTPRDGAR |
| FGF-CX1f | NTYSSNIYKHGDTGRRYFVALNKDGTPRDGARSKRHQKFTHFLPRPVDPERVPELYKDLL |
| FGF-CX1g | -------------------------------------------RSMAPLAEVGGFLGGLE |
| FGF-CX1h | MNDKGELYGSEKLTSECIFREQFEENWYNTYSSNIYKHEDTGRRYFVALNKDGTPRDGAR |
| FGF-CX1i | MNDKGELYGSEKLTSECIFREQFEENWYNTYSSNIYKHGDTGRRYFVALNKDGTPRDGAR |
| FGF-CX1j | NTYSSNIYKHGDTGRRYFVALNKDGTPRDGARSKRHQKFTHFLPRPVDPERVPELYKDLL |
| FGF-CX1k | MNDKGELYGSEKLTSECIFREQFEENWYNTYSSNIYKHGDTGRRYFVALNKDGTPRDGAR |
| FGF-CX1l | NTYSSNIYKHGDTGRRYFVALNKDGTPRDGARSKRHQKFTHFLPRPVDPERVPELYKDLL |
| FGF-CX1m | ---------------------------------------------MAPLAEVGGFLGGLE |
| FGF-CX1n | ------------------------------------------ERPPLLGERRSAAERSAR |
| FGF-CX1o | ------------------------------------------RRYFVALNKDGTPRDGAR |
| FGF-CX1p | -------------------------------------------PRPVDPERVPELYKDLL |
| FGF-CX1q | ------------------------------------------------MNDKGELYGSEK |
| FGF-CX1r | MMDKGELYGSEKLTSECIFREQFEENWYNTYSSNIYKHGDTGRRYFVALNKDGTPRDGAR |
| FGF-CX1a | SKRHQKFTHFLPRPVDPERVPELYKDLLMYT----------------------------- |
| FGF-CX1b | SKRHQKFTHFLPRPVDPERVPELYKDLLMYT----------------------------- |
| FGF-CX1c | GLGQPGAAQLAHLHGILRRRQLYCRTGFHLQILPDGSAQGTRQDHSLFGILEFISVAVGL |
| FGF-CX1d | GLGQPGAAQLAHLHGILRRRQLYCRTGFHLQILPDGSVQGTRQDHSLFGILEFISVAVGL |
| FGF-CX1e | SKRHQKFTHFLPRPVDG------------------------------------------- |
| FGF-CX1f | MYTVDG------------------------------------------------------ |
| FGF-CX1g | GLGQPGAAQLAHLHGILRRRQLYCRTGFHLQILPDGSVQGTRQDHSLFGILEFISVAVGL |
| FGF-CX1h | SKRHQKFTHFLPRPLE-------------------------------------------- |
| FGF-CX1i | SKRHQKFTHFLPRPLE-------------------------------------------- |
| FGF-CX1j | MYT--------------------------------------------------------- |
| FGF-CX1k | SKRHQKFTHFLPRPVDPERVPELYKDLLMYT----------------------------- |
| FGF-CX1l | MYT--------------------------------------------------------- |
| FGF-CX1m | GLG--------------------------------------------------------- |
| FGF-CX1n | GGP--------------------------------------------------------- |
| FGF-CX1o | SKR--------------------------------------------------------- |
| FGF-CX1p | MYT--------------------------------------------------------- |
| FGF-CX1q | LTSEC------------------------------------------------------- |
| FGF-CX1r | SKRHQKFTHFLPRPVDPERVPELYKNLLMYT----------------------------- |
| FGF-CX1a | ------------------------------------------------------------ |
| FGF-CX1b | ------------------------------------------------------------ |
| FGF-CX1c | VSIRGVDSGLYLGMNDKGELYGSEKLTSECIFREQFEENWYNTYSSNIYKHGDTGRRYFV |
| FGF-CX1d | VSIRGVDSGLYLGMNDKGELYGSEKLTSECIFREQFEENWYNTYSSNIYKHGDTGRRYFV |
| FGF-CX1e | ------------------------------------------------------------ |
| FGF-CX1f | ------------------------------------------------------------ |
| FGF-CX1g | VSIRGVDSGLYLGMNDKGELYGSEKLTSECIFREQFEENWYNTYSSNIYKHGDTGRRYFV |
| FGF-CX1h | ------------------------------------------------------------ |
| FGF-CX1i | ------------------------------------------------------------ |
| FGF-CX1j | ------------------------------------------------------------ |
| FGF-CX1k | ------------------------------------------------------------ |
| FOF-ex1l | ------------------------------------------------------------ |
| FGF-CX1m | ------------------------------------------------------------ |
| FGF-CX1n | ------------------------------------------------------------ |
| FGF-CX1o | ------------------------------------------------------------ |
| FGF-CX1p | ------------------------------------------------------------ |
| FGF-CX1q | ------------------------------------------------------------ |
| FGF-CX1r | ------------------------------------------------------------ |
| FGF-CX1a | ----------------------------------------------- |
| FGF-CX1b | ----------------------------------------------- |
| FGF-CX1c | ALNKDGTPRDGARSKRHQKFTHFLPRPVDPERVPELYKDLLMYT--- |
| FGF-CX1d | ALNKDGTPRDGARSKRHQKFTHFLPRPVDPERVPELYKDLLMYTVDG |
| FGF-CX1e | ----------------------------------------------- |
| FGF-CX1f | ----------------------------------------------- |
| FGF-CX1g | ALNKDGTPRDGARSKRHQKFTHFLPRPVDPERVPELYKDLLMYTLE- |
| FGF-CX1h | ----------------------------------------------- |
| FGF-CX1i | ----------------------------------------------- |
| FGF-CX1j | ----------------------------------------------- |
| FGF-CX1k | ----------------------------------------------- |
| FGF-CX1l | ----------------------------------------------- |
| FGF-CX1m | ----------------------------------------------- |
| FGF-CX1n | ----------------------------------------------- |
| FGF-CX1o | ----------------------------------------------- |
| FGF-CX1p | ----------------------------------------------- |
| FGF-CX1q | ----------------------------------------------- |
| FGF-CX1r | ----------------------------------------------- |
| FGF-CX1a | (SEQ ID NO: 2) |
| FGF-CX1b | (SEQ ID NO: 4) |
| FGF-CX1c | (SEQ ID NO: 6) |
| FGF-CX1d | (SEQ ID NO: 8) |
| FGF-CX1e | (SEQ ID NO: 10) |
| FGF-CX1f | (SEQ ID NO: 12) |
| FGF-CX1g | (SEQ ID NO: 14) |
| FGF-CX1h | (SEQ ID NO: 16) |
| FGF-CX1i | (SEQ ID NO: 18) |
| FGF-CX1j | (SEQ ID NO: 20) |
| FGF-CX1k | (SEQ ID NO: 22) |
| FGF-CX1l | (SEQ ID NO: 24) |
| FGF-CX1m | (SEQ ID NO: 26) |
| FGF-CX1n | (SEQ ID NO: 28) |
| FGF-CX1o | (SEQ ID NO: 30) |
| FGF-CX1p | (SEQ ID NO: 32) |
| FGF-CX1q | (SEQ ID NO: 34) |
| FGF-CX1r | (SEQ ID NO: 36) |

Further analysis of the FGF-CX1a protein yielded the following properties shown in Table 1C.

TABLE 1C
Protein Sequence Properties FGF-CX1a

| | |
|---|---|
| SignalP analysis: | No Known Signal Sequence Predicted |

PSORT II analysis:

| PSG: | a new signal peptide prediction method | |
|---|---|---|
| | N-region: length 6; pos.chg 0; | neg.chg 1 |
| | H-region: length 8; peak value | 0.00 |
| | PSG score: −4.40 | |

| | |
|---|---|
| GvH: | von Heijne's method for signal seq. recognition |
| | GvH score (threshold: −2.1): −5.49 |
| | possible cleavage site: between 16 and 17 |

>>> Seems to have no N-terminal signal peptide

| ALOM: | Klein et al's method for TM region allocation | | | |
|---|---|---|---|---|
| | Init position for calculation: 1 | | | |
| | Tentative number of TMS(s) for the threshold 0.5: 1 | | | |
| | Number of TMS(s) for threshold 0.5: 1 | | | |
| | INTEGRAL | Likelihood = | −6.42 | Transmembrane |
| | 94-110 | | | |

| | | |
|---|---|---|
| PERIPHERAL | Likelihood = | 5.20 (at 1) |

| | ALOM score: −6.42 (number of TMSs: 1) |
|---|---|
| MTOP: | Prediction of membrane topology (Hartmann et al.) |
| | Center position for calculation: 101 |
| | Charge difference: 0.5 C(0.0) − N(−0.5) |
| | C > N: C-terminal side will be inside |

>>> membrane topology: type 1b (cytoplasmic tail 94 to 211)
MITDISC: discrimination of mitochondrial targeting seq

| | | | |
|---|---|---|---|
| R content: | 0 | Hyd Moment(75): | 3.24 |
| Hyd Moment(95): | 6.56 | G content: | 4 |
| D/E content: | 2 | S/T content: | 0 |
| Score: −9.30 | | | |

Gavel: prediction of cleavage sites for mitochondrial preseq

cleavage site motif not found

NUCDISC: discrimination of nuclear localization signals

pat4: none
pat7: none
bipartite: none
content of basic residues: 12.3%
NLS Score: −0.47

KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:

type 1: none
type 2: none

NMYR: N-myristoylation pattern: none
Prenylation motif: none
memYQRL: transport motif from cell surface to Golgi: none
Tyrosines in the tail: too long tail
Dileucine motif in the tail: found

LL at 207

checking 63 PROSITE DNA binding motifs: none
checking 71 PROSITE ribosomal protein motifs: none
checking 33 PROSITE prokaryotic DNA binding motifs: none

| | |
|---|---|
| NNCN: | Reinhardt's method for Cytoplasmic/Nuclear discrimination |
| | Prediction: cytoplasmic |
| | Reliability: 89 |
| COIL: | Lupas's algorithm to detect coiled-coil regions |
| | total: 0 residues |

Final Results (k = 9/23):

```
    34.8%: nuclear
    21.7%: mitochondrial
    21.7%: cytoplasmic
      8.7%: vesicles of secretory system
      4.3%: vacuolar
      4.3%: peroxisomal
      4.3%: endoplasmic reticulum
```

>> prediction for CG53135-05 is nuc (k = 23)

A search of the FGF-CX1a protein against the Geneseq database, a proprietary database that contains sequences published in patents and patent publication, yielded several homologous proteins shown in Table 1D.

TABLE 1D
Geneseq Results for FGF-CX1a

| | | FGF- | | |
|---|---|---|---|---|
| | | CX1a | Identities/ | |
| | | Residues/ | Similarities for | |
| Geneseq | Protein/Organism/Length [Patent #, | Match | the Matched | Expect |
| Identifier | Date] | Residues | Region | Value |
| ABP54435 | Human fibroblast growth factor (FGF) | 1 . . . 211 | 211/211 (100%) | e−123 |
| | CX protein - Homo sapiens, 211 aa. | 1 . . . 211 | 211/211 (100%) | |
| | [WO200277266-A2, 03-OCT-2002] | | | |
| ABP54434 | Xenopus XFGF-CX amino acid | 1 . . . 211 | 211/211 (100%) | e−123 |
| | sequence SEQ ID NO: 24 - Xenopus | 1 . . . 211 | 211/211 (100%) | |
| | laevis, 211 aa. [WO200277266-A2, 03- | | | |
| | OCT-2002] | | | |
| ABP54429 | Human fibroblast growth factor (FGF) | 1 . . . 211 | 211/211 (100%) | e−123 |
| | CX protein SEQ ID NO: 2 - Homo | 1 . . . 211 | 211/211 (100%) | |
| | sapiens, 211 aa. [WO200277266-A2, | | | |
| | 03-OCT-2002] | | | |
| AAU75323 | Human fibroblast growth factor, FGF- | 1 . . . 211 | 211/211 (100%) | e−123 |
| | CX - Homo sapiens, 211 aa. | 1 . . . 211 | 211/211 (100%) | |
| | [WO200202625-A2, 10-JAN-2002] | | | |
| ABB07261 | Human FGF-20 polypeptide - Homo | 1 . . . 211 | 211/211 (100%) | e−123 |
| | sapiens, 211 aa. [WO200192522-A2, | 1 . . . 211 | 211/211 (100%) | |
| | 6-DEC-2001] | | | |

In a BLAST search of public sequence databases, the FGF-CX1a protein was found to have homology to the proteins shown in the BLASTP data in Table 1E.

TABLE 1E
Public BLASTP Results for FGF-CX1a

| | | FGF-CX1a | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q9NP95 | Fibroblast growth factor-20 (FGF- | 1 . . . 211 | 211/211 (100%) | e−122 |
| | 20) - Homo sapiens (Human), 211 | 1 . . . 211 | 211/211 (100%) | |
| | aa. | | | |
| Q8C7A8 | Fibroblast growth factor 20 - Mus | 1 . . . 211 | 201/211 (95%) | e−117 |
| | musculus (Mouse), 211 aa. | 1 . . . 211 | 204/211 (96%) | |
| Q9EST9 | FGF-20 - Rattus norvegicus (Rat), | 1 . . . 211 | 201/211 (95%) | e−117 |
| | 212 aa. | 1 . . . 211 | 204/211 (96%) | |
| Q9ESL9 | Fibroblast growth factor 20 - Mus | 1 . . . 211 | 200/211 (94%) | e−116 |
| | musculus (Mouse), 212 aa. | 1 . . . 211 | 204/211 (95%) | |
| Q9PVY1 | XFGF-20 - Xenopus laevis | 1 . . . 211 | 170/211 (80%) | 5e−97 |
| | (African clawed frog), 208 aa. | 1 . . . 208 | 189/211 (89%) | |

PFam analysis predicts that the FGF-CX1a protein contains the domains shown in the Table 1F.

TABLE 1F
Domain Analysis of FGF-CX1a

| | | Identities/ | |
|---|---|---|---|
| | FGF-CX1a | Similarities | Expect |
| Pfam Domain | Match Region | for the Matched Region | Value |
| FGF | 63 . . . 194 | 83/147 (56%) | 7.4e−83 |
| | | 122/147 (83%) | |

### 5.2 EXAMPLE 2

### Proteolytic Cleavage Products of CG53135-05 (FGF-20)

Liquid Chromatography, Mass spectrometry and N-terminal sequencing of CG53135-05 resulted in variants that have high activity in the proliferation assays. Thus these variants detailed in this section are expected to have same utility as that of CG53135-05.

Liquid Chromatography (LC) and Mass Spectrometry (S) Analysis of CG53135-05

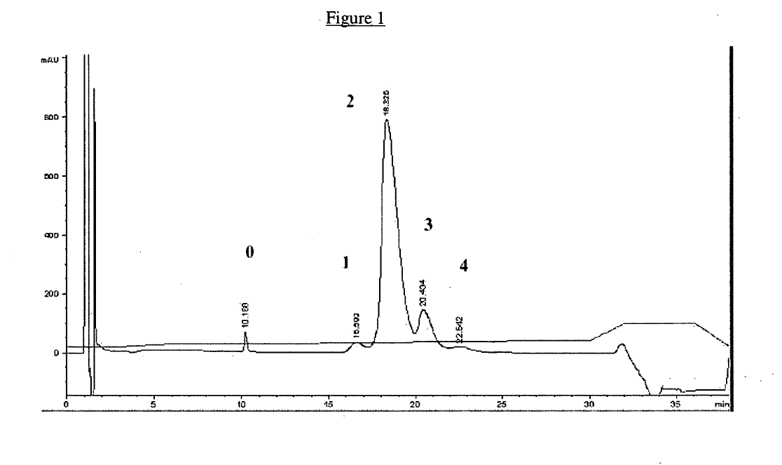

Purified CG53135-05 was injected onto a phenyl-hexyl column (Luna 5 mm, 250 mm×3 mm, Phenomenex) using a standard HPLC system (Agilent 1100, Agilent) in a mobile phase containing acetonitrile, water and trifluoroacetic acid. The resulting analysis revealed detectable levels of micro-heterogeneity, showing 1 major peak (#3) and 3 minor peaks (#1, 2, 4, and 5) related to CG53135-05 (FIG. 1). In order to characterize these species, fractions were collected using an automated fraction-collector (Agilent 1100) and the fractions characterized by liquid chromatography electrospray ionization ion trap mass spectrometry (LC/ESI/MS), matrix-assisted laser desorption ionization time of flight mass spectrometry (MALDI-TOF-MS), and N-terminal amino acid sequencing.

Peak 0 is not a protein, is not product-related, and is not a residual process impurity (i.e., it is not DNA, endotoxin, kanamycin, or IPTG). The CG53135-05-related species (Peaks 1, 2, 3, and 4) eluted in mobile phase of 36% acetonitrile, 63% water, and 0.1% trifluoroacetic acid. Similar fractions were then pooled and the samples concentrated using an Amicon 10,000 dalton cut-off filter (Millipore, Bedford, Mass.) in a 4° C. centrifuge. The samples were diluted 16-fold in 200 mM arginine, 40 mM sodium acetate, and 3% glycerol and then concentrated to a volume of approximately 500 μl. The concentration of the pooled fractions was determined using amino acid analysis.

All four CG53135-05-related species (Peaks 1, 2, 3, and 4) were proteolytically digested using trypsin and the peptides analyzed using liquid chromatography with mass spectrometry detection. Mass Spectrometry was performed using a XP DECA nanospray/ion trap instrument (ThermoFinnigan, San Jose, Calif.) interfaced with an Ultimate Nanoflow Chromatography System (LC Packings, Amsterdam, Netherlands). Data were collected via Xcalibur Software (Thermofinngan) using automated MS to MS/MS switching. In the Instrument Method files, the XP DECA was set to acquire a full MS scan between 400 and 1400 m/z followed by full MS/MS scans between 400 and 2000 m/z of the top 3 ions from the preceding MS scan. Data were processed using TurboSequest (Thermofinngan). Database searching and protein identification was performed using MASCOT (Matrix Sciences, Manchester, UK). MASCOT reports a probability-based MOWSE score and percent coverage for the identified protein based on molecular mass of the peptides, MS/MS sequence information, mass accuracy, and number of peptides detected. Table 2 contains the peak number for the CG53135-05-related species, confidence score provide by MASCOT, and percent coverage obtained from MS/MS spectra.

TABLE 2
Characterization of CG53135-05 (DEV10) by MASCOT

| | Retention | % of Total | Probability-based | % Coverage |
|---|---|---|---|---|
| Peak # | Time | Peak Area | MOWSE score | by MS/MS |
| 0 | 10.13 | 1.12 | NA | NA |
| 1 | 16.29 | 2.84 | 434 | 46% |
| 2 | 18.03 | 79.94 | 562 | 59% |
| 3 | 20.17 | 13.59 | 708 | 80% |
| 4 | 22.25 | 2.50 | 428 | 54% |

NA = data not available

The fractions collected were analyzed by MALDI-TOF and N-terminal sequencing to determine the identity of CG53135-05-related species. N-terminal amino acid sequence of purified CG53135-05 was determined qualitatively. CG53135-05 protein was resolved by SDS-PAGE and electrophoretically transferred to a polyvinylidenefluoride membrane; the Coomassie-stained ˜23 kDa major band was excised from the membrane and analyzed by an automated Edman sequencer (Procise, Applied Biosystems, Foster City, Calif.). Table 3 presents the molecular weight obtained for each species and variant determined by N-terminus sequencing (where N=full-length CG53135):

TABLE 3
Characterization of CG53135-05 (DEV10) by MALDI-TOF and
Sequence Analysis

| | | Variant |
|---|---|---|
| | Molecular Weight | (determined by N-terminal |
| Peak # | (by MALDI-TOF) | sequence) |
| 0 | NA | NA |
| 1 | 21343 | N-23 |
| | | N-14 |
| | 22380 | N-11 |
| | | N-8 |
| 2 | 23247 | N-2 |
| 3 | 23473 | N |
| 4 | 23300 | N-1 |

NA = data not available, not a protein, not product-related

The molecular weight determined by MALDI-TOF and N-terminus sequencing can identify the 4 species. For peak 1, 4 different species were detected via N-terminal sequencing, 2 of which were also detected by MALDI-TOF. These results are also in agreement with the coverage obtained using LC/MS. The polypeptide sequences of each species derived by N-terminal sequencing are given in Table 4.

TABLE 4

Polypeptide Sequences of the proteolytic cleavage products

N-8:

(SEQ ID NO:37)

GFLGGLEGLGQQVGSHFLLPPAGERPPLLGERRSAAERSARGGPGAAQLAHL
HGILRRRQLYCRTGFHLQILPDGSVQGTRQDHSLFGILEFISVAVGLVSIRGVDSGLYLG
MNDKGELYGSEKLTSECIFREQFEENWYNTYSSNIYKHGDTGRRYFVALNKDGTPRDGAR
SKRHQKFTHFLPRPVDPERVPELYKDLLMYT
N-11:

(SEQ ID NO:38)

GGLEGLGQQVGSHFLLPPAGERPPLLGERRSAAERSARGGPGAAQLAHL
HGILRRRQLYCRTGFHLQILPDGSVQGTRQDHSLFGILEFISVAVGLVSJRGVDSGLYLG
MNDKGELYGSEKLTSECIFREQFEENWYNTYSSNIYKHGDTGRRYFVALNKDGTPRDGAR
SKRHQKFTHFLPRPVDPERVPELYKDLLMYT
N-14:

(SEQ ID NO:39)

EGLGQQVGSHFLLPPAGERPPLLGERRSAAERSARGGPGAAQLAHL
HGILRRRQLYCRTGFHLQILPDGSVQGTRQDHSLFGILEFISVAVGLVSIRGVDSGLYLG
MNDKGELYGSEKLTSECIFREQFEENWYNTYSSNIYKHGDTGRRYFVALNKDGTPRDGAR
SKRHQKFTHFLPRPVDPERVPELYKDLLMYT
N-23:

(SEQ ID NO:40)

HFLLPPAGERPPLLGERRSAAERSARGGPGAAQLAHL
HGLLRRRQLYCRTGFHLQILPDGSVQGTRQDHSLFGILEFISVAVGLVSIRGVDSGLYLG
MNDKGELYGSEKLTSECIFREQFEENWYNTYSSNIYKHGDTGRRYFVALNKDGTPRDGAR
SKRHQKFTHFLPRPVDPERVPELYKDLLMYT

[image]

Total Amino Acid Analysis

The amino acid composition of CG53135-05 was determined. Samples of CG53135-05 were hydrolyzed for 16 h at 115° C. in 100 mL of 6 N HCR, 0.2% phenol containing 2 nmol norleucine as an internal standard. Samples were dried in a Speed Vac Concentrator and dissolved in 100 mL sample buffer containing 2 nmol homoserine as an internal standard. The amino acids in each sample were separated on a Beckman Model 7300 ion-exchange instrument. The amino acid composition of CG53135-05 was consistent with the theoretical amino acid composition.

The experimental amino acid composition was used to derive the extinction coeffeicient used in estimation of concentration via UV absorbance (protein estimation using the Bradford method). The extinction coefficient at λmax is 0.97 mL/mg-cm.

TABLE 6
Amino Acid Analysis of CG53135-05 (DEV10)

| | Theoretical | Experimental |
|---|---|---|
| Amino Acid | Mole Percent | Mole Percent |
| ala | 5.69 | 5.69 |
| arg | 9.00 | ND^{A} |
| asx | 6.63 | 6.68^{D} |
| cys | 0.95 | ND^{B} |
| gly | 12.80 | 13.46 |
| glx | 10.90 | 8.67^{D} |
| his | 3.79 | 4.69 |
| ile | 3.32 | 3.46 |
| leu | 12.32 | 13.18 |
| lys | 3.32 | 3.55 |
| met | 1.42 | 0.80 |
| phe | 4.74 | 4.93 |
| pro | 5.69 | 5.69 |
| ser | 6.16 | 4.98 |
| thr | 3.79 | 5.97 |
| trp | 0.47 | ND^{C} |
| tyr | 4.26 | 4.55 |
| val | 4.74 | 5.26 |

^{A}Not determined because of excess arg in the formulation;
^{B}Not determined because cys is destroyed in the acid hydrolysis during analysis;
^{C}Not determined because trp is destroyed in the acid hydrolysis during analysi;
^{D}During acid hydrolysis asn will be converted to asp and gln to glu acid. Therefore, asx represents the sum of asn and asp while glx represents the sum of gln and glu.

Peptide Mapping

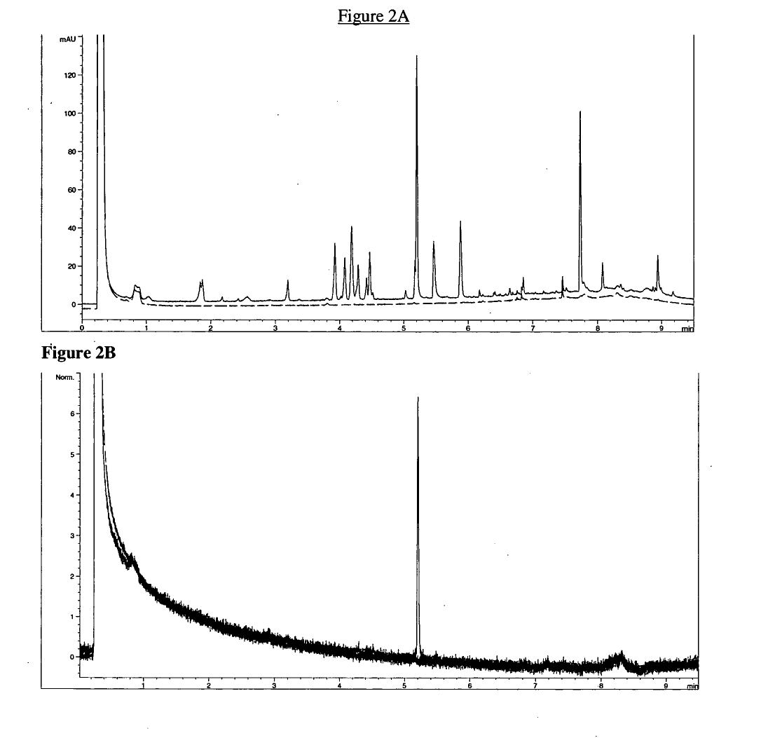

Purified CG53135-05 (25 mg) was denatured and reduced in urea and dithiothreitol at 50° C. and then alkylated with iodoacetate. After lowering the concentration of urea, the samples were treated with trypsin for 40 h at 20° C. The resulting peptide fragments were separated by RP-HPLC (using a C-18 column with an acetonitrile gradient in trifluoroacetate) to obtain a peptide map (FIGS. 2A and 2B). The chromatogram in FIG. 2A is consistent with the 20 peptides expected from the digestion of CG53135-05 with trypsin, and the chromatogram in FIG. 2B reveals a single peak as expected for the single tryptophan residue in CG53135-05.

Bioassay

The biological activity of CG53135-05 related species collected from the 4 peaks identified by LC and MS was measured by treatment of serum-starved cultured NIH 3T3 murine embryonic fibroblast cells with various doses of the isolated CG53135-05 related species and measurement of incorporation of bromodeoxyuridine (BrdU) during DNA synthesis. For this assay, cells were cultured in Dulbecco's modified Eagle's medium supplemented with 10% fetal bovine serum. Cells were grown in 96-well plates to confluence at 37° C. in 10% CO₂/air and then starved in Dulbecco's modified Eagle's medium for 24-72 h. CG53135-05-related species were added and incubated for 18 h at 37° C. in 10% CO₂/air. BrdU (10 mM final concentration) was added and incubated with the cells for 2 h 37° C. in 10% CO₂/air. Incorporation of BrdU was measured by enzyme-linked immunosorbent assay according to the manufacturer's specifications (Roche Molecular Biochemicals, Indianapolis, Ind.).

Peak 4 was not included in this assay since insufficient material was collected (Peak 4 is less than 3% of the total peak area for CG53135-05). CG53135-05 and material collected from all 3 remaining fractions (i.e., Peak 1, 2, and 3) induced DNA synthesis in NIH 3T3 mouse fibroblasts in a dose-dependent manner (Table 7). The PI₂₀₀ was defined as the concentration of protein that resulted in incorporation of BrdU at 2 times the background. CG53135-05 and CG53135-05 related species recovered from all 3 measurable peaks demonstrated similar biological activity with a PI₂₀₀ of 0.7-11 ng/mL (Table 9).

TABLE 7
Biological Activity of CG53135-05 (DEV10): Induction of DNA
Synthesis

| | CG53135-05 | | | |
|---|---|---|---|---|
| | (DEV 10 | Peak 1 | Peak 2 | Peak 3 |
| PI₂₀₀ (ng/mL) | 1.0 | 0.7 | 11 | 8.6 |

### 5.3. EXAMPLE 3

### Receptor Binding Specificity of CG53135 (Study L-116.01)

FGF family members transduce signals intracellularly via high affinity interactions with cell surface immunoglobulin (Ig) domain-containing tyrosine kinase FGF receptors (FGFRs). Four distinct human genes encode FGFRs (Powers et al., Endocr Relat Cancer 2000, 7:165-97; Klint and Claesson-Welsh, Front Biosci 1999, 4:D165-77; Xu et al., Cell Tissue Res 1999, 296:33-43). A related fifth human sequence lacking a kinase domain has recently been identified and named FGFR-5 (Kim et al., Biochim Biophys Acta 2001, 1518:152-6). These receptors can each bind several different members of this family (Kim et al., Biochim Biophys Acta 2001, 1518:152-6; Ornitz et al., J Biol Chem 1996, 271:15292-7). FGFs also bind, albeit with low affinity, to heparin sulfate proteoglycans (HSPGs) present on most cell surfaces and extracellular matrices (ECM). Interactions between FGFs and HSPGs serve to stabilize FGF/FGFR interactions and to sequester FGF and protect it from degradation (Powers et al., Endocr Relat Cancer 2000, 7:165-97; Szebenyi and Fallon, Int Rev Cytol 1999, 185:45-106). Dimerization of FGF receptor monomers upon ligand binding is reported to be a requisite for activation of the kinase domains, leading to receptor trans-phosphorylation. FGF receptor-1 (FGFR-1), which shows the broadest expression pattern of the four FGF receptors, contains at least seven tyrosine phosphorylation sites. A number of signal transduction molecules are affected by binding with different affinities to these phosphorylation sites.

FGFR-1, FGFR-2 and FGFR-3 each recognize FGF-1, FGF-2, FGF-4 and FGF-8. In addition, FGFR-1 & FGFR-2 bind FGF-3, FGF-5, FGF-6, FGF-10 and FGF-17 (Powers et al., Endocr Relat Cancer 2000, 7:165-97). Binding of various FGF ligands varies with each receptor splice form, thus allowing a wide repertoire of FGF-mediated signaling events through a limited number of receptor coding genes. Tissue-specific alternate splicing permits cells expressing a single FGFR gene to significantly diversify their biological response by generating distinct receptor isoforms that may exhibit different ligand specificity and function. FGFR-4, binds FGF-1, FGF-2, FGF-4, FGF-6, FGF-8 and FGF-9 but not FGF-3, FGF-5 or FGF-7. FGF-7, or keratinocyte growth factor-1 (KGF-1) is only recognized by FGFR-2, whereas FGF-9 binds to FGFR-2, FGFR-3 and FGFR-4. Receptor specificity of FGFs-11 to -19 is not well understood (Powers et al., Endocr Relat Cancer 2000, 7:165-97; Ornitz et al., J Biol Chem 1996, 271:15292-7).

Immunohistochemistry studies (Hughes, J Histochem Cytochem 1997, 45:1005-19) in normal human adult tissues from the major organ systems indicated that FGFR-1, FGFR-2 and FGFR-3 are widely expressed, suggesting an important functional role in tissue homeostasis. Protein expression patterns for tissue-specific isoforms have not yet been determined. FGFR-4 has a more limited expression pattern being notably absent from lung, oviduct, placenta, testis, prostate, thyroid, parathyroid, and sympathetic ganglia, tissues where all three other receptors are predominantly expressed (Hughes, J Histochem Cytochem 1997, 45:1005-19).

To determine the receptor binding specificity of CG53135, we examined the effect of soluble FGFRs on the induction of DNA synthesis in NIH 3T3 cells by recombinant CG53135-01 produced in E. coli.

Materials and Methods

Protein Purification from Escherichia coli: For production in E. coli, plasmid pETMY-hFGF20X was transformed into the E. coli expression host BL21 (Novagen, Madison, Wis.) and the induction of protein CG53135 expression was carried out according to the manufacturer's instructions. pETMYhFGF20X/BL21 E. coli bacteria were grown in LB medium at 37° C. At an OD of 0.6, bacteriophage lambda (CE6) was added to a final multiplicity of infection of 5. The infected culture was further incubated at 27° C. for 3 hours. After induction, total cells were harvested, and proteins were analyzed by Western blotting using anti-HisGly antibody (Invitrogen). Cells were harvested by low-speed centrifugation (5000 rpm in a GS-3 rotor for 15 minutes at 4° C.), suspended in phosphate-buffered saline (PBS) containing 0.5M NaCl and 1M arginine, and disrupted with two passes through a microfluidizer. Cell debris was removed by low-speed centrifugation and the soluble protein fraction (supernatant) was clarified by filtration through a 0.2 micron low-protein binding membrane. The protein sample was then loaded onto a metal chelation column (pre-charged with nickel sulfate). The nickel column was washed with PBS/0.5M NaCl+1M L-arginine and bound proteins were eluted with a linear gradient of imidazole (0-0.5 M). Fractions containing CG53135 (100-150 mM imidazole) were pooled and dialyzed against 1×10⁶ volumes of PBS pH 8.0 containing 1M L-arginine. The protein sample was stored at −80° C.

Receptor Specificity: NIH 3T3 cells were cultured in 96-well plates to approximately 100% confluence, washed and fed with DMEM without supplements (Life Technologies), and incubated for 24 h. Recombinant CG53135-01 or control protein was then added to the cells for 18 h. Control proteins used were aFGF (positive control) and platelet derived growth factor-BB (PDGFBB) (negative control). To analyze the effect of soluble FGFRs on CG53135 activity, recombinant CG53135-01, aFGF, or PDGF-BB (final concentrations of 10, 5 and 3 ng/mL, respectively), were mixed with soluble receptors (final concentrations of 0.2, 1 and 5 ug/mL), and incubated for 30 min at 37° C. prior to addition to serum-starved NIH 3T3 cells. Factor concentrations represent the amount of ligand needed to generate a half maximal BrdU response in NIH 3T3 cells. Soluble FGFRs were Fc chimeras of the following receptor forms (FGFR1β(IIIc); FGFR2β(IIIb); FGFR2α(IIIb); FGFR2α(IIIc); FGFR3α(IIIc); FGFR4) and were obtained from R&D Systems (Minneapolis, Minn.). The BrdU assay was performed according to the manufacturer's specifications (Roche Molecular Biochemicals, Indianapolis, Ind.) using a 4 h BrdU incorporation time.

Results and Conclusions

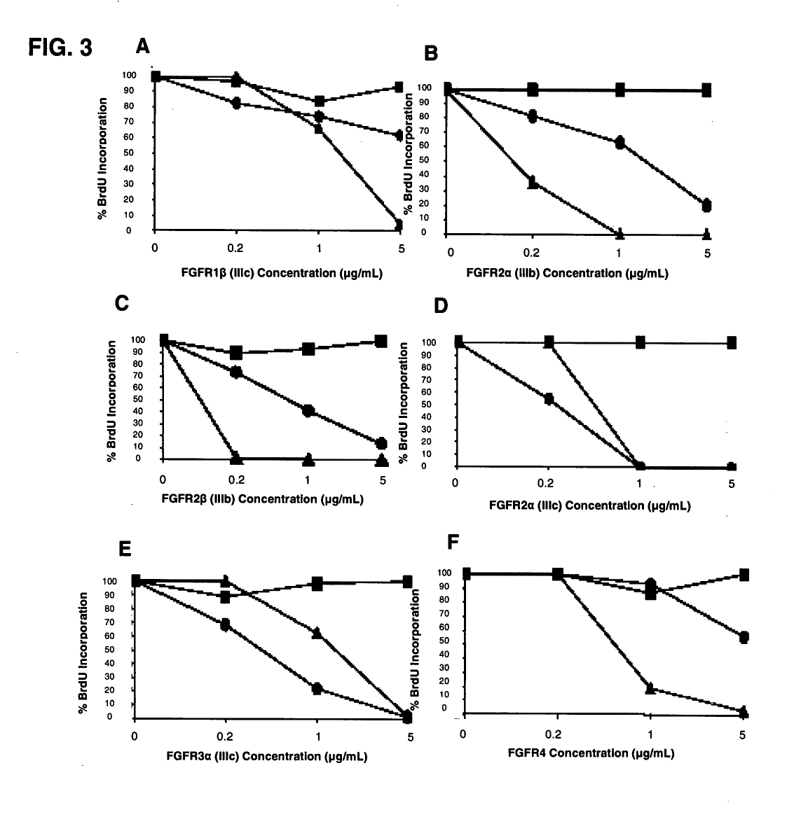

To determine the receptor binding specificity of CG53135, we examined the effect of soluble FGFRs on the induction of DNA synthesis in NIH 3T3 cells by recombinant CG53135-01 produced in E. coli. Soluble receptors for FGFR1β(IIIc), FGFR2β(IIIb), FGFR2α(IIIb), FGFR2α(IIIc), FGFR3α(IIIc), and FGFR4 were utilized. We found that soluble forms of each of these FGFRs were able to specifically inhibit the biological activity of CG53135 (FIG. 3).

Complete or nearly complete inhibition was obtained with soluble FGFR2α(IIIb), FGFR2β(IIIb), FGFR2a (IIIc), and FGFR3α (IIIc), whereas partial inhibition was achieved with soluble FGFR1β (IIIc) and FGFR4. None of the soluble receptor reagents interfered with the induction of DNA synthesis by PDGF-BB (FIG. 2), thereby demonstrating their specificity. The integrity of each soluble receptor reagent was demonstrated by showing their ability to inhibit the induction of DNA synthesis by aFGF, a factor known to interact with all of the FGFR's under analysis (FIG. 3).

### 5.4. EXAMPLE 4

### Treatment of Stroke

Thirty male Sprague Dawley rats were allocated to treatment groups as indicated in the study design Table 8 below.

TABLE 8
Experimental Design

| | Number of | | |
|---|---|---|---|
| | Animals | Dose* | Volume* |
| Treatment | Males | (μg) | (μL) |
| Vehicle 1 | 10 | 0 | 50 |
| CG53135-05 | 10 | 1 | 50 |
| CG53135-05 | 10 | 2.5 | 50 |

*Administered dose and volume is based on an average bodyweight of 330 g.

Experimental Procedures

Middle cerebral artery (MCA) Surgery and Intracisternal Injections: Animals were handled for 7 days prior to surgery. Cefazolin sodium (40 mg/kg, i.p) was administered on the day before surgery and just after surgery. At the time of surgery, the rats were anesthetized with 2% halothane in a 2:1 N₂O:O₂ mixture. Body temperature was maintained at 37±0.50° C. The proximal right MCA was electrocoagulated from just proximal to the olfactory tract to the inferior cerebral vein and was then transected. For intracisternal injections, animals were re-anesthetized as above and placed in a stereotaxic frame. Rats were given CG53135-05 or vehicle [40 mM acetate, 200 mM mannitol (pH 5.3)] by percutaneous injection into the cisterna magna, once at 1 day, (approximately 24 hours) and once at 3 days, (approximately 72 hours) after MCA. Animals were given test article (2 dose groups) or vehicle treatment according to the study design.

Clinical Observations/Signs

Animals were observed immediately over a 1 hour period following injections for signs of seizure (indicated by tremor and violent motion about the cage), pain (indicated by loud vocalization), and lethargy. Animals were also observed daily for mortality and moribundity.

Body Weight: Animals were weighed on Days 1, 3, 7, 14 and 21.

Limb Placing Test: limb placing tests were carried out on all animals on Day −1 (pre-operation), Day 1 (just prior to injection), Day 3 and then every 7 days thereafter (Days 7, 14, 21).

Forelimb Placing Test Assessment Score: The forelimb placing test measures sensorimotor function in each forelimb as the animal places the limb on a table top in response to visual, tactile, and proprioceptive stimuli. The forelimb placing test consists of the following evaluations and scoring, where the combined total score for the forelimb placing test reflects a range from 0 (no impairment) to 10 (maximal impairment):
visual placing (forward, sideways): 0-4
tactile placing (dorsal, lateral): 0-4
proprioceptive placing: 0-2
Total score for all forelimb tests: 0-10

Hindlimb Placing Test Assessment Score: Similarly, the hindlimb placing test measures sensorimotor function of the hindlimb as the animal places it on a tabletop in response to tactile and proprioceptive stimuli. The hindlimb placing test consists of the following evaluations and scoring, where the combined total score for the hindlimb placing test reflects a range from 0 (no impairment) to 6 (maximal impairment):
tactile placing (dorsal, lateral): 0-4
proprioceptive placing: 0-2
Total score for all hindlimb tests: 0-6

Body Swing Test: the body swing test was carried out on all animals on Day −1 (pre-operation), Day 1 (just prior to injection), Day 3 and then every 7 days thereafter (Days 7, 14, 21).

The body swing test examines side preference as the animal is held approximately one inch above the surface of the table, and swings to the right or the left side. Thirty swings are counted, and the score is then calculated based on the percentage of swings to the right. (score range=˜50% right swing (no impairment)-0% right swing (maximal impairment))

Cylinder Test: the cylinder test was carried out on all animals on Day −1 (pre-operation) and 7 days thereafter (Days 7, 14, 21). The cylinder test measures spontaneous motor activity of the forelimbs. Animals are placed in a narrow glass cylinder (16.5×25 cm) and videotaped for 5 min on the day before stroke surgery and at weekly intervals thereafter. Videotapes are then scored independently by one experienced observer and up to 50 spontaneous movements will be counted (−5 min per rat per day). Spontaneous movements include those made by each forelimb to initiate rearing, to land on or to move laterally along the wall of the cylinder, or to land on the floor after rearing.

Macroscopic and Histomorphology: on the day of scheduled termination (Day 3), animals were euthanized by an intraperitoneal injection of Chloral hydrate (500 mg/Kg). Brains were examined grossly and removed, postfixed in formalin, dehydrated and embedded in paraffin. Coronal sections (5 mm) will be cut on a microtome mounted on to glass slides, and stained with hematoxylin/eosin (H&E). The area of cerebral infarcts on each of seven slices (+4.7, +2.7, +0.7, −1.3, −3.3, −5.3, and −7.3 mm compared with Bregma) was determined using a computer interface imaging system using the indirect method (area of the intact contralateral hemisphere−area of the intact ipsilateral hemisphere) to correct for brain shrinkage during processing. Infarct volume was then expressed as a percentage of the intact contralateral hemispheric volume. Volumes of the infarction in the cortex and striatum were also determined separately using these same methods. H&E stained section was examined for histological changes such as hemorrhage, abscess or tumor formation.

Statistical Analysis: all intracisternal injections, behavioral testing, and subsequent histological analyses were done by investigators blinded to the treatment assignment of each animal. Data are then expressed as means+/−SEM, and will be analyzed by one or two way (ANOVA) followed by appropriate pairwise post hoc tests with correction for multiple comparisons.

Results

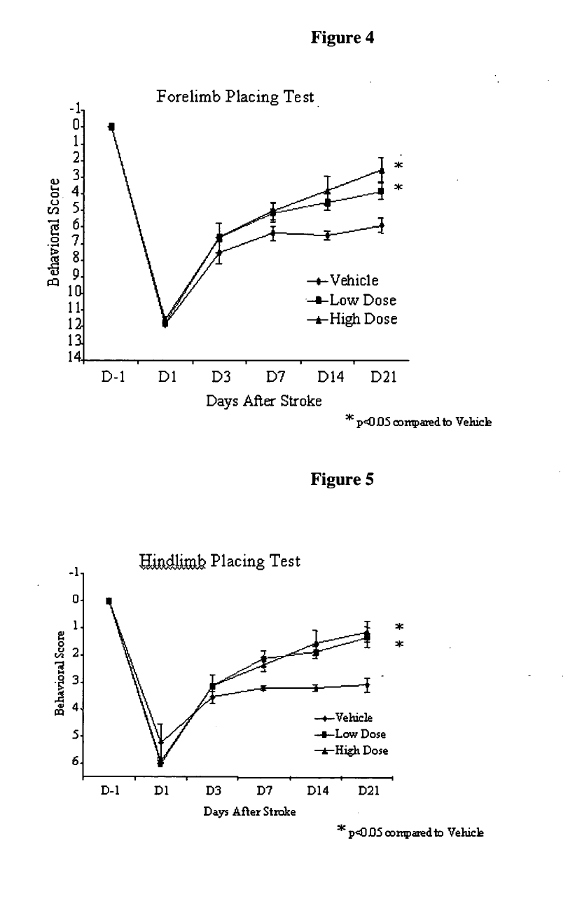

Forelimb Placing Test: on days −1, 1, 3, 7, 14, and 21 relative to MCA occlusion, animals were examined by using a limb placing test to assess sensorimotor function in the forelimb in response to visual, tactile and proprioceptive stimuli (Kawamata, T., Dietrich, W. D., Schallert, T., Gotts, E., Cocke, R. R., Benowitz, L. I. & Finklestein, S. P. (1997) Proc. Natl. Acad. Sci. USA 94, 8179-8184; De Ryck, M., Van Reempts, J., Duytschaever, H., Van Deuren, B. & Clincke, G. (1992) Brain Res. 573, 44-60.) Visual placing (scored 04), tactile placing (scored 0-4), and proprioceptive placing (scored 0-2) were summed to generate a range of potential total scores from 0 to 12, with 12 representing maximal impairment (FIG. 4).

Hindlimb Placing: on days −1, 1, 3, 7, 14, and 21 relative to MCA occlusion, animals were examined by using a limb placing test to assess sensorimotor function in the hindlimb in response to tactile and proprioceptive stimuli [Kawamata, T., Dietrich, W. D., Schallert, T., Gotts, E., Cocke, R. R., Benowitz, L. I. & Finklestein, S. P. (1997) Proc. Natl. Acad. Sci. USA 94, 8179-8184; De Ryck, M., Van Reempts, J., Duytschaever, H., Van Deuren, B. & Clincke, G. (1992) Brain Res. 573, 44-60]. Tactile placing (scored 04), and proprioceptive placing (scored 0-2) were summed to generate a range of potential total scores from 0 to 6, with 6 representing maximal impairment (FIG. 5).

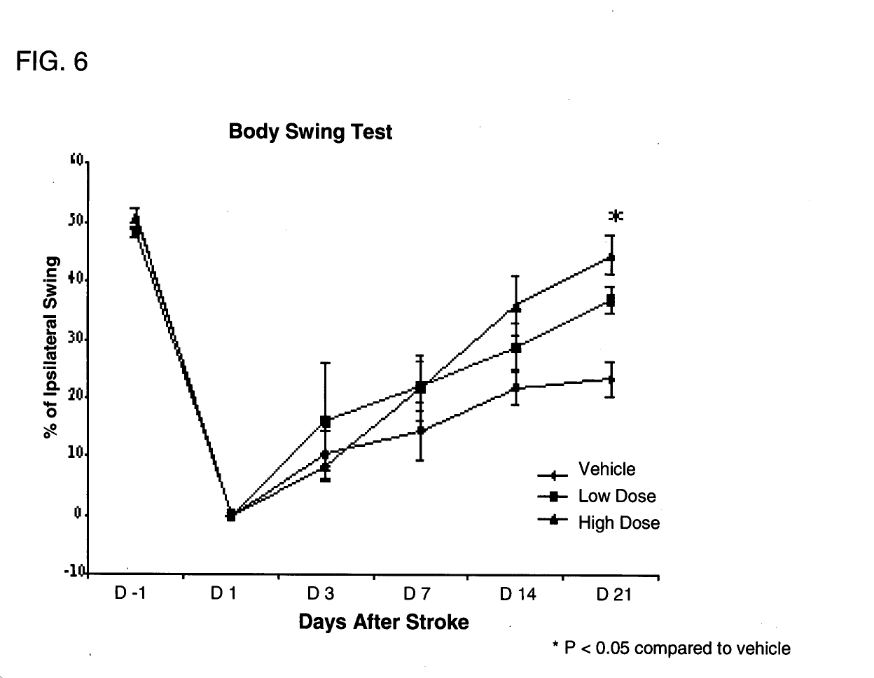

Body Swing Test: On days −1, 1, 3, 7, 14, and 21 relative to MCA occlusion, animals were examined by using a body swing test to assess side preference as the animal is held approximately one inch above the surface of the table, and swings to the right or the left side. (Kawamata, T., Dietrich, W. D., Schallert, T., Gotts, E., Cocke, R. R., Benowitz, L. I. & Finklestein, S. P. (1997) Proc. Natl. Acad. Sci. USA 94, 8179-8184; De Ryck, M., Van Reempts, J., Duytschaever, H., Van Deuren, B. & Clincke, G. (1992) Brain Res. 573, 4460.) Thirty swings were counted, and the score calculated based on the percentage of swings to the right (FIG. 6).

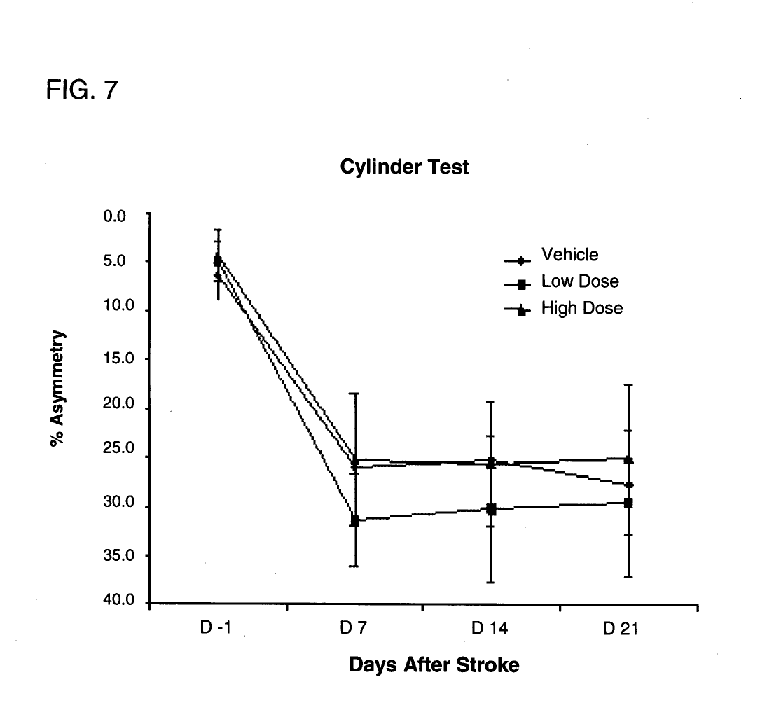

Cylinder Test: On days −1, 1, 3, 7, 14, and 21 relative to MCA occlusion, animals were examined by cylinder test to assess spontaneous motor activity of the forelimbs (Kawamata, T., Dietrich, W. D., Schallert, T., Gotts, E., Cocke, R. R., Benowitz, L. I. & Finklestein, S. P. (1997) Proc. Natl. Acad. Sci. USA 94, 8179-8184; De Ryck, M., Van Reempts, J., Duytschaever, H., Van Deuren, B. & Clincke, G. (1992) Brain Res. 573, 44-60.) Briefly, animals are placed in a narrow glass cylinder (16.5×25 cm) and videotaped for 5 min on the day before stroke surgery and at weekly intervals thereafter. Videotapes are then scored independently by one experienced observer and up to 50 spontaneous movements will be counted (˜5 min per rat per day). Spontaneous movements include those made by each forelimb to initiate rearing, to land on or to move laterally along the wall of the cylinder, or to land on the floor after rearing (FIG. 7).

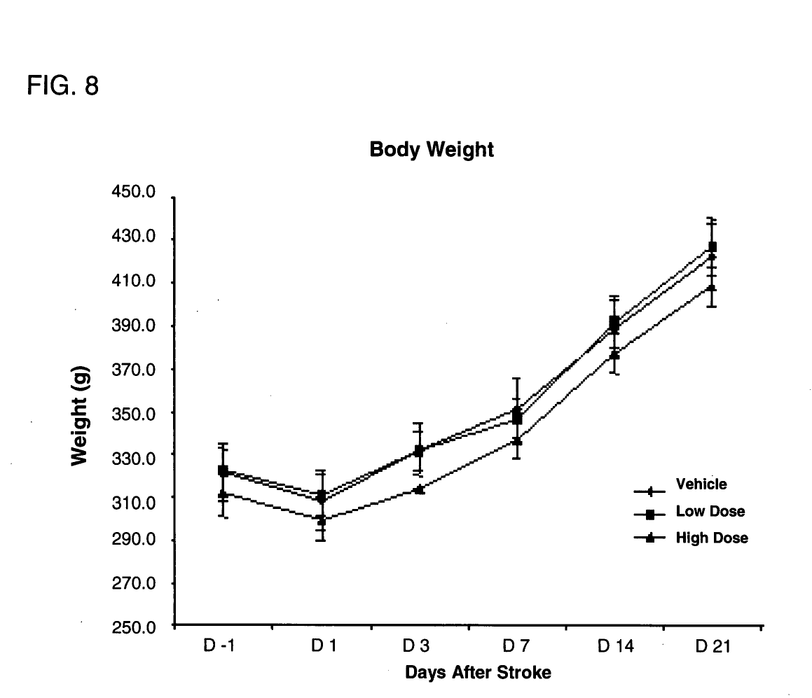

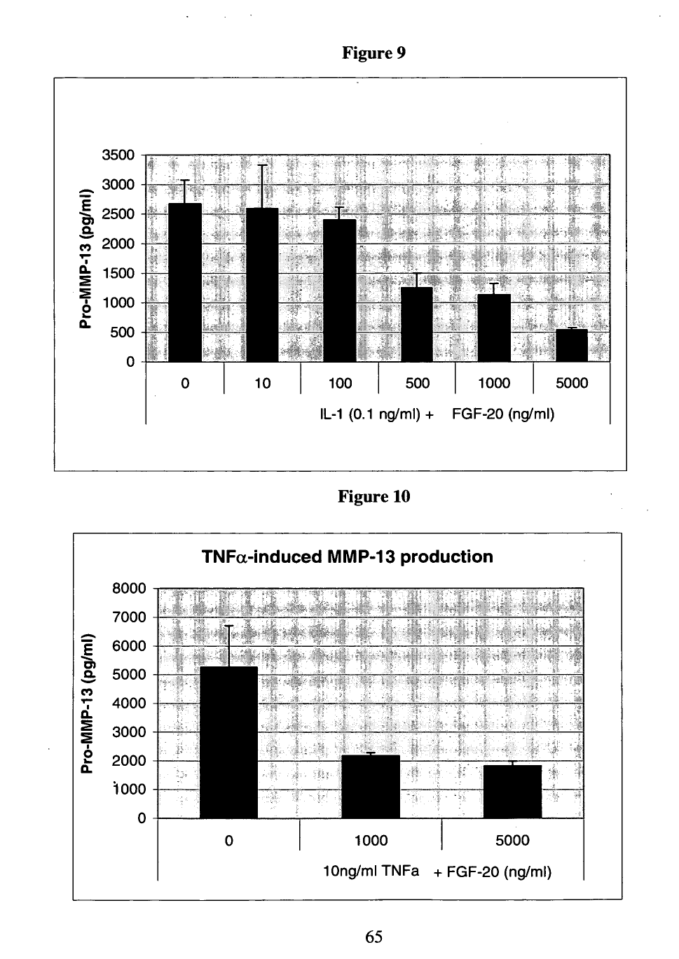

Body Weight: animals were weighed on days −1, 1, 3, 7, 14, and 21 relative to MCA occlusion and the results indicate no significant difference between the vehicle and CG53135-05 treatment (FIG. 8).

Conclusion

Administering CG53135-05 following MCA occlusion suggested that both the low and high doses produced a significant enhancement of recovery on forelimb (FIG. 4) and hindlimb placing tests (FIG. 5) for the contralateral (affected) limbs, and improvement on the body swing test (FIG. 6). This pattern of activity with other therapeutics in this model has generally been shown to reflect improvement in cerebrocortical and subcortical (striatal) function, respectively (Dijkhuizen R M, Ren J, Mandeville J B, Wu O, Ozdag F M, Moskowitz M A, Rosen B R, Finklestein S P. 2001, Proc Natl Acad Sci USA 98(22):12766-71). No apparent differences were seen on the cylinder test (FIG. 7) of spontaneous limb use or on animal body weight (FIG. 8).

Therefore, CG53135-05 administration will be useful in the treatment of pathological conditions including ischemic stroke, hemorrhagic stroke, trauma, spinal cord damage, heavy metal or toxin poisoning and neurodegenerative diseases (such as Alzheimer's, Parkinson's Disease, Amyotrophic Lateral Sclerosis, Huntington's Disease).

### 5.5. EXAMPLE 5

### Matrix Metalloproteinase Production Assay

The matrix metalloproteinases (MMPs) are a family of related enzymes that degrade the extracellular matrix in bone and cartilage. These enzymes operate during normal development in tissues differentiation and remodeling. In arthritic diseases, such as Osteoarthritis (OA) and Rheumatoid Arthritis (RA), elevated expression of these enzymes contribute to irreversible matrix degradation. Thus, effect of CG53135-05 on MMP production was assayed.

The activity of CG53135 on matrix metalloproteinase (MMP) production was assessed using the SW1353 chondrosarcoma cell line (ATCC HTB-94). This cell line is a well-established chondrocytic cellular model for matrix metalloproteinases (NMP) production. SW1353 cells were plated in a 24-well plate at 1×10⁵ cells/ml (1 ml) in DMEM medium—10% FBS. Following overnight incubation, the medium was replaced with DMEM+0.2% Lactabulmin serum. CG53135-05 was added to the wells at doses ranging from 10 to 5000 ng/ml, in the absence or presence of IL-1 beta (0.1 to 1 ng/ml, R&D systems Minneapolis, Minn.), TNF-alpha (10 ng/ml, R&D systems) or vehicle control to a final volume of 0.5 ml. IL-1 beta and TNF-alpha are both potent stimulators of MMP activity. All treatments were done in triplicate wells. After 24 h, the supernatants were collected and Pro-MMP-1, and -13, as well as TIMP-1 (tissue inhibitor of matrix metalloproteinase), a natural inhibitor of MMP activity, was measured by ELISA (R&D systems). The measurements were normalized to the number of cells by an MTS assay.

Results

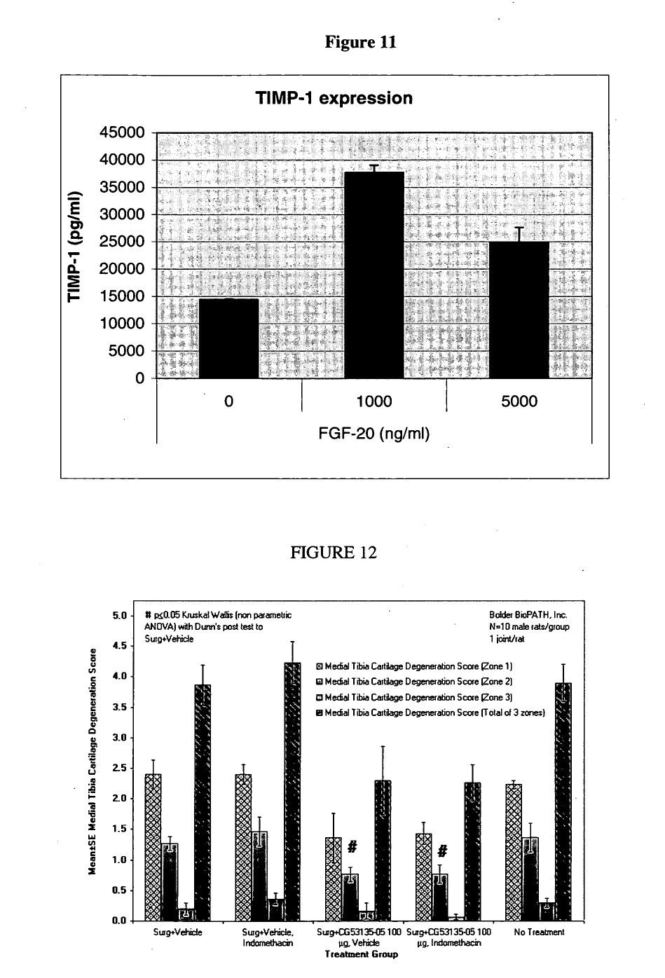

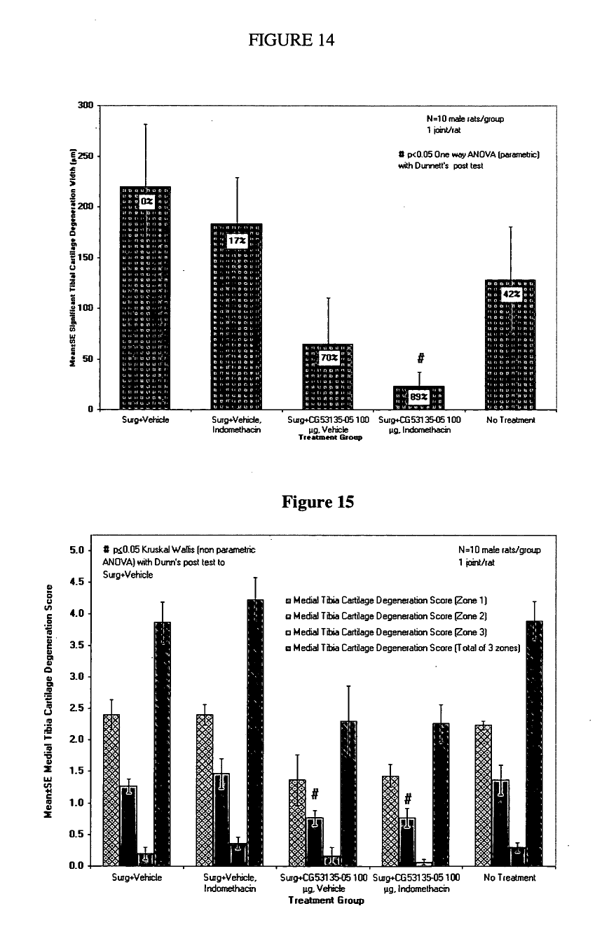

CG53135-05 significantly decreased MMP-13 production in the presence of either IL-1 beta or TNF-alpha as demonstrated in FIG. 14 and FIG. 15 respectively. IL-1 beta and TNF-alpha are both potent stimulators of MMP activity. MMP-13 affinity for type II collagen, the main collagen that is degraded in OA, is ten times higher that of MMP-1. Since MMP-13 expression increases in OA and RA, the decrease of MMP-13 observed with addition of CG53135-05 indicates that the protein can be used as an OA and RA therapeutic (FIG. 10). Furthermore, CG53135-05 up-regulated the production of TIMP-1, a natural inhibitor of MMP activity (FIG. 11). This enhancement of TIMP-1 production by CG53135-05 is beneficial in reducing the matrix breakdown by MMP-1 and -13 observed in OA and RA. In addition, CG53135-05 had no effect on MMP-3 production constitutively or after IL-1 induction (data not shown.). Similarly, CG53135-05 (FGF-20) showed increase in basal expression of MMP-1 in SW1353 cells (data not shown).

### 5.6. EXAMPLE 6

### Effect of CG53135-05 on Normal Rats: Proof of Principle to the Meniscal Tear Model

The effect of CG53135-05 on the normal rats was studied as a proof of principle to drive further studies in disease model (ex: meniscal tear model of osteoarthritis in rats). The effect of CG53135-05 on synovium and cartilage was assessed by injecting the protein into normal male Lewis rats.

Effects of Intra-Articular Injection of CG53135-05 in Normal Rats

The rats were injected intra-articularly three times per week for 2 weeks with vehicle solution (8 mM acetate, 40 mM arginine, and 0.6% glycerol (pH 5.3) in approximately 1% hyaluronic acid), 10 μg CG 53135-05 or 100 μg CG 53135-05.

Study Design: Male Lewis rats weighing 293-325 grams on day 0 were obtained from Harlan Sprague Dawley (Indianapolis, Ind.) and acclimated for 8 days. The rats were divided into three treatment groups with three animals in each group: two groups received CG53135 and one received only the vehicle control. The rats were anesthetized with Isoflurane and injected through the patellar tendon into the area of the cruciate attachments of both knees. CG53135 was injected at doses of 0.1 mg/ml (0.01 mg/joint) or 1.0 mg/ml (0.1 mg/joint). Controls were injected with the vehicle solution as described above. Injections were done Monday, Wednesday and Friday for 2 weeks. The animals were terminated on day 15 at which time they were injected ip with BRDU (100 mg/kg) in order to pulse label proliferating cells.

Observations and Analysis of Markers of Pathology

Gross observations Rats were observed daily for abnormal swelling or gait alterations and were weighed weekly.

Histopathology Preserved and decalcified (5% formic acid) knees were trimmed into 2 approximately equal longitudinal (ankles) or frontal (knees) halves, processed through graded alcohols and a clearing agent, infiltrated and embedded in paraffin, sectioned, and stained with toluidine blue (knees). Multiple sections (3 levels) of right knee were analyzed microscopically with attention to the parameters of interest listed below. Each parameter was graded as normal, minimal, mild, moderate, marked or severe. Evaluation of the cartilage was done using descriptive parameters rather than the scoring criteria generally used in the osteoarthritis model because of the type of alterations generated by the repetitive injection of the protein. Although animals were injected with BRDU prior to termination, the proliferative changes were readily observed in toluidine blue stained sections.

Results

TABLE 9
Microscopically Monitored Parameters

| | | Central Cruciate | |
|---|---|---|---|
| | Cartilage | Attachment Area | |
| Synovial Alterations | Alterations | Alterations | Chondrogenesis |
| hyperplasia | cartilage | inflammation and | marginal zone or |
| infiltration of | proteoglycan | fibroplasia | periosteal |
| synovium with | loss | bone or cartilage | chondrogenesis |
| macrophages | | | |
| fibroplasia | cartilage | damage | |
| matrix | fibrillation | | |
| (proteoglycan | | | |
| deposition in | | | |
| fibrotic synovium) | | | |

Live Phase Parameters Body weights were similar in vehicle and protein injected animals throughout the study (Table 12). Knees injected with 100 μg of protein had some evidence of fibrosis clinically during the injection process beginning with the 3rd injection.

Morphologic Pathology Vehicle injected rats had minimal to mild synovial hyperplasia, inflammation and fibroplasia with none to minimal matrix deposition in fibrotic synovium. Articular cartilage had no proteoglycan loss or fibrillation. The central area of the joint where the cruciates attach and in which the intra-articular injections are made had none to minimal fibroplasia and cartilage/bone damage. No marginal zone chondrogenesis was present.

Knees injected with 10 μg CG 53135-05 had mild to moderate synovial hyperplasia, inflammation and fibroplasia with minimal to moderate matrix deposition in fibrotic synovium. Articular cartilage had no proteoglycan loss or fibrillation. The central area of the joint where the cruciates attach and in which the intra-articular injections are made had none to minimal fibroplasia and cartilage/bone damage. One knee had minimal marginal zone chondrogenesis.

Knees injected with 100 μg CG 53135-05 had moderate to marked synovial hyperplasia, inflammation and fibroplasia with moderate matrix deposition in fibrotic synovium. Articular cartilage had none to minimal proteoglycan loss or fibrillation. The central area of the joint where the cruciates attach and in which the intra-articular injections are made had minimal to marked fibroplasia and cartilage/bone damage. All knees had mild to moderate marginal zone chondrogenesis. One animal had chondrogenesis in areas associated with articular cartilage.

Conclusion

These results demonstrate that repetitive intra-articular injection of CG53135-05 induces synovial fibroplasia and chondrogenesis. Vehicle injections resulted in mild inflammation and fibroplasia thus suggesting that this vehicle has some irritant potential. Concentration responsive increases in synovial proliferative response as well as marginal zone chondrogenesis occurred in animals injected with protein. The area of the cruciate attachment where injections occurred had areas of bone resorption and fibroplasia which also increased in severity with increasing concentrations of the protein as did the synovial inflammation. The potentially adverse effects of observed synovial fibroplasia and bone resorption could have been due to either FGF-20 activity or endotoxin levels within the non-clinical grade hyaluronic acid used to formulate the protein. In addition, inflammation in the joint can induce bone resorption and marginal zone chondrogenesis so these results need to be interpreted in light of the possibility that the inflammatory response to the protein injection contributed to the proliferative response. The morphologic appearance of the proliferative changes and chondrogenesis clearly indicates that the biological activity of this protein (CG53135-05) is important in generating the response.

The results of the experiments reported herein indicate repetitive intra-articular injection of CG53135-05 induces synovial fibroplasia and chondrogenesis.

### 5.7. EXAMPLE 7

### Intra-Articular Injection of CG53135-05 in Meniscal Tear Model of Rat Osteoarthritis: Prophylactic and Therapeutic Dosing

Example 6 utilized CG53135-05 administration into the joints of normal rats to identify effects on relevant cell populations by histomorphometric analysis. At the dose of 100 ug/joint, CG53135-05 induced significant marginal zone chondrogenesis similar to that seen with other growth factors such as TGF-beta, suggesting an effect on pluripotent stem cells within the marginal zone. There was no apparent effect on mature chondrocytes as evidenced by the lack of a response in the mature cartilage areas of the joints. The potentially adverse effects of observed synovial fibroplasia and bone resorption could have been due to either FGF-20 activity or endotoxin levels within the non-clinical grade hyaluronic acid used to formulate the protein.

Further studies in osteoarthritic animals performe addressed the following: 1) synergy with an anti-inflammatory drug (standard approach for osteoarthritis patients), 2) whether CG53135-05 (FGF-20) can induce functional repair or protection of joint cartilage layers, and 3) whether synovial fibroplasia and bone resorption were FGF-20-induced or due to contaminating endotoxin within the formulation.

Thus one aspect of this study was to evaluate the protective and therapeutic effects of intra-articular injection of CG53135-05 on joint damage in osteoarthritis in the meniscal tear model of rat osteoarthritis. This relatively new model of OA has been shown to have morphologic alterations of cartilage degeneration and osteophyte formation that resemble changes occurring in spontaneous disease and surgically induced disease in other species (Bendele, A. M., Animal Models of Osteoarthritis. J. Musculoskel. Neuron Interact. 2001; 1:363-376, Bendele, A. M. and Hulman, J. F. Spontaneous cartilage degeneration in guinea pigs. Arthritis Rheum. 1988; 31:561-565). The model can be used to evaluate potential beneficial effects of anti-degenerative as well as regenerative therapies.

Experimental Design

Animals (10/group), housed 2/cage, were anesthetized with Isoflurane and the right knee area is prepared for surgery. A skin incision was made over the medial aspect of the knee and the medial collateral ligament was exposed by blunt dissection, and then transected. The medial meniscus was then reflected medially with a fine scissor and a cut was made through the full thickness to simulate a complete tear. The skin was closed with suture.

Prophylactic Dosing: intra-articular dosing (CG53135-05) of the right knee joint was initiated on the day of surgery and is continued for 2 weeks post-surgery with intra-articular injections given Thursday, Saturday, and Monday (day 0, 2, 4, 7, 9, and 11) with rats under Isoflurane anesthesia. Indomethacin, a nonsteroidal anti-inflammatory drug, was dosed (1 mg/kg/day) daily by the oral route starting on the day of surgery to reduce any potential inflammation due to the injection. Body weights were recorded on days 0, 7 and 14. After animal termination on day 14 post-surgery, both knees were collected for histopathologic evaluation. The study design is shown in Table 10.

TABLE 10
Prophylactic Dosing Study Design

| | | | Number of |
|---|---|---|---|
| | CG53135-05 | Co-therapy | Animals |
| Group | Treatment^{a} | Treatment^{b} | Males |
| 1 | Vehicle | Vehicle | 10 |
| | (intra-articular) | | |
| 2 | Vehicle | Indomethacin | 10 |
| | (intra-articular) | | |
| 3 | CG53135-05 | Vehicle | 10 |
| | (intra-articular) | | |
| 4 | CG53135-05 | Indomethacin | 10 |
| | (intra-articular) | | |
| 5 | None | None | 10 |

^{a}Administration 3 times per week for 2 weeks (100 μg/joint, intra-articular)
^{b}Administration daily for 2 weeks (0.5 mg/kg, PO)

Therapeutic Dosing: intra-articular dosing (CG53135-05) of the right knee joint is initiated on day 21 of post-surgery and is continued for 2 weeks with intra-articular injections given Friday, Sunday, and Tuesday (day 22, 25, 27, 29, 32, and 34) with rats under Isoflurane anesthesia. Indomethacin is dosed daily by the oral route starting on the day of surgery. Body weights are recorded on days 0, 7, 14, 21, 28, and 35. On day 35, both knees are collected for histopathologic evaluation. The study design is shown in Table 11.

TABLE 11
Therapeutic Dosing Study Design

| | | | Number of |
|---|---|---|---|
| | CG53135-05 | Co-therapy | Animals |
| Group | Treatment^{a} | Treatment^{b} | Males |
| 1 | Vehicle | Vehicle | 10 |
| | (intra-articular) | | |
| 2 | Vehicle | Indomethacin | 10 |
| | (intra-articular) | | |
| 3 | CG53135-05 | Vehicle | 10 |
| | (intra-articular) | | |
| 4 | CG53135-05 | Indomethacin | 10 |
| | (intra-articular) | | |
| 5 | None | None | 10 |

^{a}Administration 3 times per week for 2 weeks (100 μg/joint, intra-articular)
^{b}Administration daily for 2 weeks (0.5 mg/kg, PO)

Results of prophylactic dosing study: Observations made include the standards followed for this model. Multiple sections (3 levels) of right knee were analyzed microscopically and scored according to the following methods. In scoring the 3 sections, the worst case scenario for the 2 halves on each of the 3 slides representing 3 levels was determined for cartilage degeneration and osteophyte formation. This value for each parameter for each slide was then averaged to determine overall subjective cartilage degeneration scores for tibia and femur and osteophyte scores for tibia.

Cartilage degeneration was scored none to severe (numerical values 0-5) for depth and area (surface divided into thirds) using the following criteria:
0=no degeneration
1=minimal degeneration, chondrocyte and proteoglycan loss with or without fibrillation involving the superficial zone
2=mild degeneration, chondrocyte and proteoglycan loss with or without fibrillation involving the upper ⅓
3=moderate degeneration, chondrocyte and proteoglycan loss with fibrillation extending well into the midzone and generally affecting ½ of the total cartilage thickness
4=marked degeneration, chondrocyte and proteoglycan loss with fibrillation extending well into the deep zone but without complete (to the tidemark) loss of matrix
5=severe degeneration, matrix loss to tidemark

Strict attention to zones (outside, middle, inside thirds) was adhered to in this scoring method and the summed scores reflect a global summation of severity of tibial degeneration.

In addition to this overall subjective analysis of cartilage degeneration, an additional subjective assessment was done using similar criteria to evaluate severity of degeneration but with attention to specific regional differences across the tibial plateau. In this OA model, generally the outside ⅓ of the tibia is most severely affected by the meniscal tear injury with lesions often extending to the tidemark by 3 weeks post-surgery. The middle ⅓ is usually a transition zone where severe or marked change becomes moderate or mild and the inner ⅓ seldom has changes greater than mild or minimal. In an attempt to determine potential differences of treatment on the severe lesion of the outside ⅓ vs. the milder lesions of the middle ⅓ and inside ⅓, these regions were each scored separately. The sum of the regional values was calculated and expressed as sum of 3 zones.

In addition to the above subjective scoring, a micrometer measurement of total extent of tibial plateau affected by any severity of degeneration (Total Tibial Cartilage Degeneration Width μm) extended from the origination of the osteophyte or marginal zone if no osteophyte was present with adjacent cartilage degeneration (outside ⅓) across the surface to the point where tangential layer and underlying cartilage appeared histologically normal.

An additional measurement (Significant Cartilage Degeneration Width μm) reflected areas of tibial cartilage degeneration in which chondrocyte and matrix loss extended through greater than 50% of the cartilage thickness.

Finally, a micrometer depth of any type of lesion (cell/proteoglycan loss, change in metachromasia, but may have good retention of collagenous matrix and no fibrillation) expressed as a ratio of depth of changed area vs. depth to tidemark was included and taken over 4 equally spaced points on the tibial surface. These measurements were taken (1 st) matrix adjacent to osteophyte (2nd) ¼ of the distance across the tibial plateau (3rd) ½ of the distance across the tibial plateau (4th) ¾ of the distance across the tibial plateau. This measurement was the most critical analysis of any type of microscopic change present. The depth to tidemark measurement (denominator) also gives an indication of cartilage thickness across the tibial plateau and therefore allows comparisons across groups when trying to determine if hypertrophy or hyperplasia has occurred.

A single tibial growth plate measurement was taken for each section in an area thought to best represent the overall width in the non tangential plane of the section.

Scoring of the osteophytes and categorization into small, medium and large was done with an ocular micrometer.
None=0 no measurable proliferative response at marginal zone
Small osteophytes=1 (up to 299 μm)
Medium osteophytes=2 (300-399 μm)
Large osteophytes=3 (>400 μm)

The score (0-3) was included in the overall joint score. In addition, the mean±SE for the actual osteophyte measurement (average for 3 sections) was also determined.

Generally, in doing the surgery, attempts were made to transect the collateral ligament at a location that results in the meniscus being reflected proximally toward the femur. The cut was then made by inserting the scissors tip toward the femur rather than the tibia. Some mechanical damage may then be detected in the femoral condylar cartilage but is rarely encountered on the tibia, thus making the tibia the most appropriate site for assessment of chondroprotection.

Focal small areas of proteoglycan and cell loss that were likely a result of physical trauma to the femoral cartilage were described but not included in the score with larger more diffuse areas receiving subjective scores according to methods described for the tibia. These larger areas were more consistent with non traumatic degeneration. Because of the possibility of iatrogenic lesions on the femur, overall joint scores were expressed both with and without femoral cartilage degeneration scores.

Damage to the calcified cartilage layer and subchondral bone was scored using the following criteria:
0=No changes
1=Increased basophilia at tidemark: no fragmentation of tidemark or marrow changes
2=Increased basophilia at tidemark: minimal to mild fragmentation of calcified cartilage of tidemark, mesenchymal change in marrow involves ¼ of total area but generally is restricted to subchondral region under lesion
3=Increased basophilia at tidemark: Mild to marked fragmentation of calcified cartilage, Mesenchymal change in marrow is up to ¾ of total area, Areas of marrow chondrogenesis may be evident but no collapse of articular cartilage into epiphyseal bone
4=Increased basophilia at tidemark: Marked to severe fragmentation of calcified cartilage, Marrow mesenchymal change involves up to ¾ of area and articular cartilage has collapsed into the epiphysis to a depth of 250 μm or less from tidemark
5=Increased basophilia at tidemark: Marked to severe fragmentation of calcified cartilage, Marrow mesenchymal change involves up to ¾ of area and Articular cartilage has collapsed into the epiphysis to a depth of greater than 250 μm from tidemark

Descriptive comments were made on degree of synovial inflammation, synovial fibrosis, marginal zone chondrogenesis, bone resorption, fibrous overgrowth with or without chondrogenesis/incorcoration into existing cartilage

Statistical Analysis: statistical analysis of histopathologic parameters was done by comparing group means using the Student's two-tailed t-test with significance set at p≦0.05. Because of the nature of the data, a non Parametric ANOVA (Kruskal-Wallis test) was used to analyze the scored parameters and a parametric ANOVA was used to analyze the measurements. The appropriate post test used was Dunnett's multiple comparisons test on the parametric data and a Dunn's test was used on the non parametric data. Significance was set at p≦0.05 for all parameters.

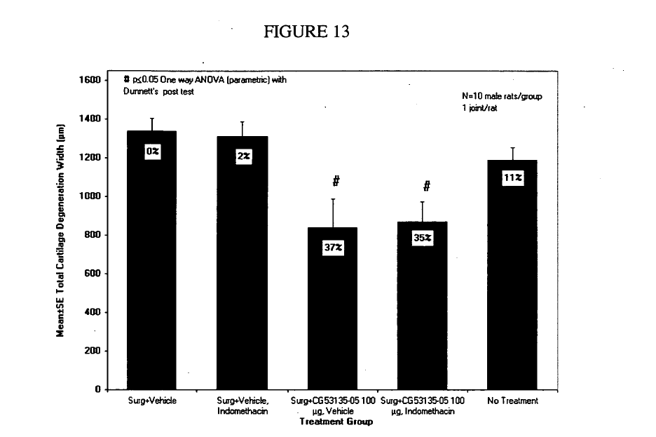

Results: intra-articular injection of 100 μg CG53135-05 with or without concurrent indomethacin administration resulted in significant inhibition (39%) of tibial cartilage degeneration on the middle ⅓ (40-43% for zone 1) and an overall insignificant inhibition of the summed 3 zones of 41% (FIG. 12). Total cartilage degeneration width was significantly decreased 35-37% (FIG. 13) and significant degeneration was reduced 70-89% with this inhibition being significant only in the group treated with protein and indomethacin (FIG. 14).

Results of the prophylactic dosing study: the data described indicate that intra-articular injection of 100 μg of CG53135-05 in knee joints of rats with medial meniscal tear results in chondroprotective effects as a result of both inhibition of cartilage degeneration and stimulation of cartilage repair. Some joints had layering of proliferated new cartilage over existing normal appearing or damaged cartilage. This observation is particularly exciting as it demonstrates the potential for resurfacing to occur.

These beneficial effects were always associated with diffuse synovial fibroplasia, bone resorption and increased synovial inflammation. Concurrent indomethacin treatment (1 mg/kg/day) had minimal if any effect on the disease process in knees injected with Synvisc alone or the disease process and reaction to the protein in knees injected with Synvisc containing protein. The single exception to this statement is reflected in the data for osteophyte measurements where all groups had similar measurements except the group treated with protein and vehicle po. This group had greater measurements thus suggesting greater marginal zone stimulation, not an uncommon occurrence in inflamed joints.

The morphologic changes induced by injection of 100 μg of this protein demonstrate the potential for CG53135-05 to be effective in cartilage repair processes. It has the capacity to induce fibrous tissue proliferation with differentiation to cartilage and importantly, integration of that newly proliferated tissue. The proliferative processes are somewhat disorganized and counter productive in areas such as the marginal zone and subchondral bone. However, rodents definitely have much greater propensity to exhibit marginal zone, periosteal and marrow proliferation from a variety of stimuli including inflammatory mediators so some of the excessive and counter productive responses seen in rats might not occur in dogs or primates. Also, there may have been some induction of an antibody response thus leading to enhanced knee inflammation that would not occur in humans or other animals that did not have an antibody response.

Additional studies that are useful in delineating the potential efficacy of CG53135-05 in osteoarthritis include:

1. Evaluation in a dog model of OA—this would allow evaluation in a larger joint with cartilage and bone structure that is more similar to humans and this is a species that has less tendency to exhibit hyperproliferative responses such as those that occur in rodents.

2. Evaluation of ia injections for 34 weeks, possibly with more aggressive anti-inflammatory systemic therapy followed by a recovery period to see how the new tissue remodels would be interesting. It may be that allowing the joint to remodel with no further proliferative stimulus would result in a more pleasing morphologic endpoint. Cycles of treatment with periods of remodeling might be the way to achieve the most satisfactory repair. Studies such as these would also answer the question of whether the repair tissue will hold up long term. Generally fibrocartilage has less of a tendency to do this.

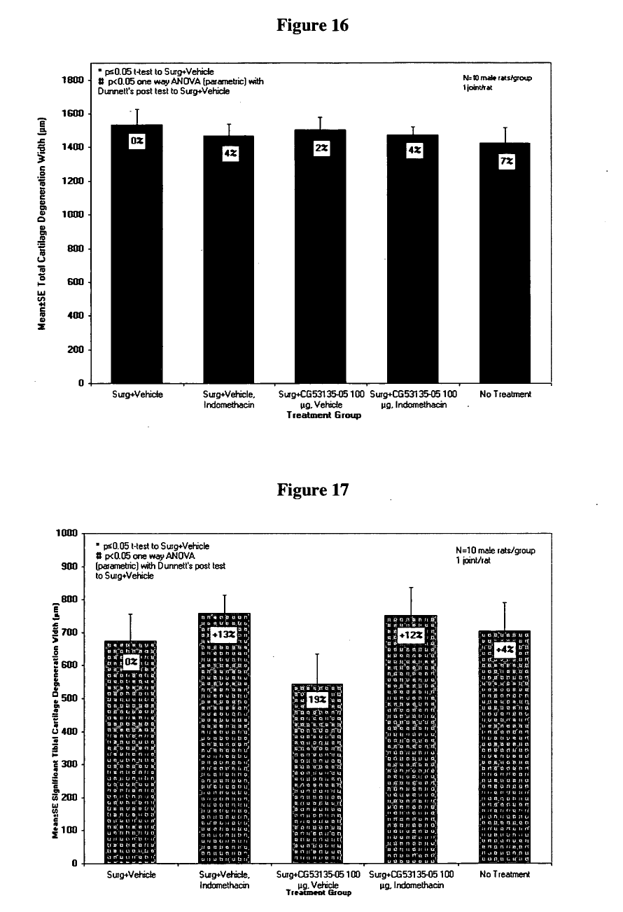

Results: Intra-articular injection of 100 μg CG53135-05 with or without concurrent oral indomethacin administration did not result in significant inhibition of tibial cartilage degeneration scores (FIG. 15). Total or significant cartilage degeneration width was not decreased (FIGS. 16, 17).

Results of the therapeutic dosing study: The data described demonstrated the potential chondroproliferative activities of CG53135-05 administered intra-articularly. However, protein injected joints had markedly increased inflammation, fibroplasia and connective tissue resorptive process.

The most important difference between the prophylactic and therapeutic dosing studies was the nature of the OA lesion at the time of initiation of dosing. Rats in the therapeutic dosing study had an area of severe matrix loss in the outer to middle ⅓ of the cartilage thus exposing the calcified cartilage/subchondral bone to the protein. Effective repair thus required filling of this defect with newly proliferated tissue coming from the marginal zone or exposed marrow pleuripotential cells. In the prophylactic dosing study, beneficial effects required inhibition of matrix degradation and stimulation of repair on a degenerating scaffold with repair tissue originating from the marginal zone only. Since the filling of a defect would be much more difficult than repairing a damaged scaffold, it may be that a longer duration of treatment would be required in a therapeutic model in order to see beneficial effects.

Indomethacin treatment was not effective in reducing the inflammatory changes and it had no beneficial effects on inhibiting the resorptive processes occurring in bone. In order to achieve effective proliferation and differentiation to cartilage in the absence of inflammation and tissue destruction, following modification to the therapeutic dosing study can be attempted: Increasing the dosing interval to once or twice weekly and/or increasing the study duration to allow time for the proliferative tissue to fill the large cartilage defects induced by this disease process. Another possibility is to investigate the effects of CG53135-05 in a larger species such as the dog as dogs have less of a tendency to proliferate connective tissue and resorb bone in response to various stimuli than rodents.

The results detailed herein (both prophylactic and therapeutic dosing studies) indicate that CG53135-05 has specific utility in severely osteoarthritic joints that are destined for joint replacement. These types of agents would be injected into joints that have little or no normal cartilage remaining and are in need of resurfacing. In this situation, repair could originate from pleuripotential cells in the marginal zones or bone marrow. Repair originating from these locations will likely result in production of fibrocartilage rather than hyaline cartilage. However, some cartilage would be preferable to no cartilage and it may be that an injectable method of sustaining a cartilage surface would be acceptable even though treatments would likely have to be repeated over time to sustain the repair. Treatments with injectable anabolic agents will likely require some kind of cyclical process in conjunction with continuous passive motion rather than sustained active load bearing motion.

### 6. Equivalence and Reference Cited

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only. We therefore do not wish to be limited to the precis terms set forth, but desire to avail ourselves of such changes and alterations that may be made for adapting the invention to various usages and conditions. Such alterations and changes may include, but not limited to, different compositions for the administration of the polypeptides according to the present invention to a subject; different amounts of the polypeptide; different times and means of administration; different materials contained in the administration dose including, for example, combinations of different peptides, or combinations of peptides with different biologically active compounds. Such changes and alterations also are intended to include modifications in the amino acid sequence of the specific polypeptides described herein in which such changes alter the sequence in a manner as not to change the functionality of the polypeptide, but as to change solubility of the peptide in the composition to be administered to a subject, absorption of the peptide by the body, protection of the polypeptide for either shelf life or within the body until such time as the biological action of the peptide is able to bring about the desired effect, and such similar modifications. Accordingly, such changes and alterations are properly intended to be within the full range of equivalents of the present invention.

1-9. (canceled)

10. A method of preventing or treating arthritis or cartilage degeneration comprising administering to a subject in need thereof an effective amount of a composition comprising an isolated protein selected from the group consisting of:
(a) a protein comprising an amino acid sequence of SEQ ID NO: 2, 6, 8, 10, 12, 14, 16, 18, 20, 24, 36, 37, 38, 39, or 40;
(b) a protein comprising amino acids 2-211 or 3-211 of SEQ ID NO: 2;
(c) a protein with one or more conservative amino acid substitutions to the protein of (a) or (b); and
(d) a fragment of the protein of (a) or (c), which fragment retains cell proliferation stimulatory activity.

11. A method of reducing matrix metalloproteinase production in a subject comprising administering to the subject an effective amount of a composition comprising an isolated protein selected from the group consisting of:
(a) a protein comprising an amino acid sequence of SEQ ID NO: 2, 6, 8, 10, 12, 14, 16, 18, 20, 24, 36, 37, 38, 39, or 40;
(b) a protein comprising amino acids 2-211 or 3-211 of SEQ ID NO: 2;
(c) a protein with one or more conservative amino acid substitutions to the protein of (a) or (b); and
(d) a fragment of the protein of (a) or (c), which fragment retains cell proliferation stimulatory activity.

12. A method of stimulating cartilage regeneration or repair comprising administering to a subject in need thereof an effective amount of a composition comprising an isolated protein selected from the group consisting of:
(a) a protein comprising an amino acid sequence of SEQ ID NO: 2, 6, 8, 10, 12, 14, 16, 18, 20, 24, 36, 37, 38, 39, or 40;
(b) a protein comprising amino acids 2-211 or 3-211 of SEQ ID NO: 2;
(c) a protein with one or more conservative amino acid substitutions to the protein of (a) or (b); and
(d) a fragment of the protein of (a) or (c), which fragment retains cell proliferation stimulatory activity.

13. A method of preventing or treating stroke or a neurodegenerative disease comprising administering to a subject in need thereof an effective amount of a composition comprising an isolated protein comprising amino acids 3-211 of SEQ ID NO: 2 or an amino acid sequence of SEQ ID NO: 6, 8, 10, 12, 14, 16, 18, 20, 24, 36, 37, 38, 39, or 40.

14. A method of preventing or treating arthritis comprising administering to a subject in need thereof an effective amount of a composition comprising an isolated protein comprising amino acids 3-211 of SEQ ID NO: 2.

15. The method of claim 14, wherein said composition further comprises an isolated protein comprising an amino acid sequence selected form the group consisting of amino acids 2-211 of SEQ ID NO: 2, and SEQ ID NOs: 2, 37, 38, 39, and 40.

16. The method of claim 14, wherein said arthritis is osteoarthritis or rheumatoid arthritis.

17. The method of claim 10, 13 or 14, wherein said composition further comprises a pharmaceutically acceptable carrier.

18. A method of claim 10 or 13, wherein said isolated protein comprises two or more proteins.

19. The method of claim 18, wherein said proteins comprise amino acid sequences selected from the group consisting of SEQ ID NOs: 2, 37, 38, 39, and 40, amino acids 2-211 of SEQ ID NO: 2 and amino acids 3-211 of SEQ ID NO: 2.

20. A method of claim 10, 13 or 14, wherein said administering is parenteral administration.

21. The method of claim 20, wherein said parenteral administration is intravenous administration.

22. The method of claim 20, wherein said parenteral administration is subcutaneous administration.

23. The method of claim 10, 13 or 14, wherein said administering is transdermal administration.

24. A method of claim 10, 13 or 14, wherein said administering is transmucosal administration.

25. The method of claim 24, wherein said transmucosal administration is nasal administration.

26. An isolated nucleic acid molecule selected from the group consisting of:
(a) a nucleic acid molecule encoding a protein comprising an amino acid sequence selected from the group consisting of SEQ ID NOs: 37, 38, 39, and 40, and amino acids 3-211 of SEQ ID NO: 2;
(b) a fragment of an nucleic acid molecule of (a), wherein said fragment encodes a protein that retains cell proliferation stimulatory activity; and
(c) a complement of an nucleic acid molecule of (a) or (b).

27. A vector comprising the nucleic acid molecule of claim 26.

28. The vector of claim 27, wherein said nucleic acid molecule is operably linked to an expression control sequence.

29. A prokaryotic or eukaryotic host cell containing the nucleic acid molecule of claim 26.

30. A prokaryotic or eukaryotic host cell containing the vector of claim 27.

31. A method comprising culturing the host cell of claim 27 in a suitable nutrient medium.

32. The method of claim 31, wherein said host cell is E. coli.

33. The method of claim 31 further comprising isolating a protein encoded by said nucleic acid molecule from said cultured cells or said nutrient medium.

34. An isolated protein by the method of claim 33.

35. An isolated protein selected from the group consisting of:
(a) a protein comprising an amino acid sequence of SEQ ID NO: 37, 38, 39 or 40;
(b) a protein consisting of amino acids 3-211 of SEQ ID NO: 2;
(c) a protein with one or more conservative amino acid substitutions to (a) or (b); and
(d) a fragment of the protein of (a)-(c) that retains cell proliferation stimulatory activity.

36. A pharmaceutical composition comprising a pharmaceutically acceptable carrier, and a protein of claim 35.


SEQUENCE LISTING (40 sequences)

SEQ ID NO: 1 - DNA, 636 bp, Homo sapiens
  CDS (1)..(633)
atggctccgctggctgaagttggtggtttcctgggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 2 - protein, 211 aa, Homo sapiens
MAPLAEVGGFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 3 - DNA, 633 bp, Homo sapiens
  CDS (1)..(633)
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacact

SEQ ID NO: 4 - protein, 211 aa, Homo sapiens; identical to SEQ ID NO: 2

SEQ ID NO: 5 - DNA, 540 bp, Homo sapiens
  CDS (1)..(537)
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgcgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttag

SEQ ID NO: 6 - protein, 179 aa, Homo sapiens
MAPLAEVGGFLGGLEGLGQPGAAQLAHLHGILRRRQLYCRTGFHLQILPDGSAQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 7 - DNA, 556 bp, Homo sapiens
  CDS (2)..(556)
caccagatctatggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacactgtcgacggc

SEQ ID NO: 8 - protein, 185 aa, Homo sapiens
TRSMAPLAEVGGFLGGLEGLGQPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYTVDG

SEQ ID NO: 9 - DNA, 415 bp, Homo sapiens
  CDS (2)..(415)
caccagatctatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtcgacggc

SEQ ID NO: 10 - protein, 138 aa, Homo sapiens
TRSILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDG

SEQ ID NO: 11 - DNA, 466 bp, Homo sapiens
  CDS (2)..(466)
caccagatctatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacactgtcgacggc

SEQ ID NO: 12 - protein, 155 aa, Homo sapiens
TRSILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYTVDG

SEQ ID NO: 13 - DNA, 549 bp, Homo sapiens
  CDS (1)..(549)
agatctatggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacactctcgag

SEQ ID NO: 14 - protein, 183 aa, Homo sapiens
RSMAPLAEVGGFLGGLEGLGQPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYTLE

SEQ ID NO: 15 - DNA, 408 bp, Homo sapiens
  CDS (1)..(408)
agatctatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatgaagacactggccgcaggtattttgtggcacttaacaaagacggaactccaagagatggcgccaggtccaagaggcatcagaaatttacacatttcttacctagaccactcgag

SEQ ID NO: 16 - protein, 136 aa, Homo sapiens
RSILRRRQLYCRTGFHLQILPDGSVQGTRQDHSLFGILEFISVAVGLVSIRGVDSGLYLGMNDKGELYGSEKLTSECIFREQFEENWYNTYSSNIYKHEDTGRRYFVALNKDGTPRDGARSKRHQKFTHFLPRPLE

SEQ ID NO: 17 - DNA, 408 bp, Homo sapiens
  CDS (1)..(408)
agatctatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccactcgag

SEQ ID NO: 18 - protein, 136 aa, Homo sapiens
RSILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPLE

SEQ ID NO: 19 - DNA, 477 bp, Homo sapiens
  CDS (1)..(474)
atggctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 20 - protein, 158 aa, Homo sapiens
MAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 21 - DNA, 636 bp, Homo sapiens
  CDS (1)..(633)
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttga

SEQ ID NO: 22 - protein, 211 aa, Homo sapiens; identical to SEQ ID NO: 2

SEQ ID NO: 23 - DNA, 540 bp, Homo sapiens
  CDS (1)..(537)
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttag

SEQ ID NO: 24 - protein, 179 aa, Homo sapiens
MAPLAEVGGFLGGLEGLGQPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 25 - DNA, 54 bp, Homo sapiens
  CDS (1)..(54)
atggctcccttagccgaagtcgggggctttctgggcggcctggagggcttgggc

SEQ ID NO: 26 - protein, 18 aa, Homo sapiens
MAPLAEVGGFLGGLEGLG

SEQ ID NO: 27 - DNA, 63 bp, Homo sapiens
  CDS (1)..(63)
gagcggccgccgctgctgggcgagcgcaggagcgcggcggagcggagcgcgcgcggcgggccg

SEQ ID NO: 28 - protein, 21 aa, Homo sapiens
ERPPLLGERRSAAERSARGGP

SEQ ID NO: 29 - DNA, 63 bp, Homo sapiens
  CDS (1)..(63)
cgcaggtattttgtggcacttaacaaagacggaactccaagagatggcgccaggtccaagagg

SEQ ID NO: 30 - protein, 21 aa, Homo sapiens
RRYFVALNKDGTPRDGARSKR

SEQ ID NO: 31 - DNA, 60 bp, Homo sapiens
  CDS (1)..(60)
cctagaccagtggatccagaaagagttccagaattgtacaaggacctactgatgtacact

SEQ ID NO: 32 - protein, 20 aa, Homo sapiens
PRPVDPERVPELYKDLLMYT

SEQ ID NO: 33 - DNA, 51 bp, Homo sapiens
  CDS (1)..(51)
atgaacgacaagggcgagctgtacggcagcgagaagctgaccagcgagtgc

SEQ ID NO: 34 - protein, 17 aa, Homo sapiens
MNDKGELYGSEKLTSEC

SEQ ID NO: 35 - DNA, 633 bp, Homo sapiens
  CDS (1)..(633)
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaagaacctactgatgtacact

SEQ ID NO: 36 - protein, 211 aa, Homo sapiens [sequence omitted]

SEQ ID NO: 37 - protein, 203 aa, Homo sapiens
GFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 38 - protein, 200 aa, Homo sapiens
GGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 39 - protein, 197 aa, Homo sapiens
EGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 40 - protein, 188 aa, Homo sapiens
HFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT